(19) United States
(12) Patent Application Publication
(10) Pub. No.: US 2026/0123867 A1
(43) Pub. Date: May 7, 2026

(54) APPARATUSES, SYSTEMS, AND METHODS FOR RAPID ON-SITE MULTISCALE MULTIMODAL DONOR ORGAN VIABILITY FACTOR CHARACTERIZATION

(71) Applicant: OrganAI Health, Inc., Chapel Hill, NC (US)

(72) Inventors: Genevieve Christine Springer, Chapel Hill, NC (US); Filip Findeyev, Laguna Niguel, CA (US)

(21) Appl. No.: 19/485,213

(22) PCT Filed: Jun. 14, 2024
(86) PCT No.: PCT/US2024/034223

§ 371 Date: Nov. 17, 2025

Related U.S. Application Data
(60) Provisional application No. 63/472,940, filed on Jun. 14, 2023.

(51) Int. Cl. A61B 5/20; A61B 5/00; G01N 29/24; G01N 29/28; G01N 29/46; G16H 10/60

(52) U.S. Cl. CPC A61B 5/201; A61B 5/0095; A61B 5/413; A61B 5/70; G01N 29/2418; G01N 29/28; G01N 29/46; G16H 10/60; G01N 2291/02475

(57) ABSTRACT

An donor organ viability instrument (1000) includes an in situ donor organ interrogation module with a positioned photoacoustic array (1008). This array, tailored for the organ type and viability factors, performs computational photoacoustic imaging to generate viability data for the donor organ (1028) in its natural location. In some implementations, the instrument includes an ex vivo donor organ interrogation module (4000) with a stationary photoacoustic array (1007). This array, also tailored for the organ type and viability factors, performs computational photoacoustic imaging and multispectral scanning on an organ placed in an acoustic coupling nest (4016). Additionally, it includes sensors for gravimetric, dimensional, and environmental measurements to assess organ viability. In certain implementations, the apparatus includes a predictive model with machine learning algorithms trained on a plurality of feature signals (6002) and corresponding organ responses (6004).

### CROSS-REFERENCES TO RELATED APPLICATIONS

The present application claims the benefit of priority to U.S. Provisional Application No. 63/472,940 filed on Jun. 14, 2023, which is herein incorporated in its entirety to the extent permissible under applicable patent laws and rules for the relevant jurisdictions.

### FIELD

The subject matter disclosed herein relates generally to technologies for ex vivo organ viability factor characterization and more particularly relates to apparatuses, systems, and methods for rapid on-site multiscale multimodal donor organ viability factor characterization.

### BACKGROUND

In the modern era, organ shortages and equitable organ allocation has led to the development of a wide variety of different approaches for characterizing donor organ viability factors. Some of these approaches are subject to variation due to differences between different individual practitioners performing various assessments and/or interpreting separately determined results of the different observations, measurements, and/or test results. Moreover, some advanced technologies for characterizing certain donor organ viability factors are typically omitted because they do not fit well in existing donor organ assessment workflows and the clinical or financial costs and/or time involved in obtaining information available via such technologies may outweigh the perceived benefits. In short, no existing system or instrument provides the benefits and advantages of the apparatuses, systems, and methods for rapid on-site multiscale multimodal donor organ viability factor characterization set forth in the present disclosure.

### BRIEF SUMMARY

One or more apparatuses, systems, and methods for rapid on-site multiscale multimodal donor organ viability factor characterization are disclosed. In some aspects, the techniques described herein relate to an apparatus including: an organ viability analysis instrument including: an in situ organ interrogation module including a positioned photoacoustic array with predetermined spectral and directional laser excitation and acoustic response signal capabilities selected based on an organ type and respective viability factors for an organ in its natural location within a body by performing photoacoustic computational imaging, the photoacoustic array configured to produce one or more first sets of organ response data related to the viability factors for the in situ organ; an ex vivo organ interrogation module including: a stationary photoacoustic array with predetermined spectral and directional laser excitation and acoustic response signal capabilities selected based on organ type and respective viability factors for an ex vivo organ received in an acoustic coupling nest, the photoacoustic array configured to produce one or more second sets of organ response data related to viability factors by performing photoacoustic, multispectral scanning; and one or more direct measurement sensors configured to perform one or more of gravimetric, dimensional, and environmental measurements related to viability factors for ex vivo organ using respective non-wave-based modalities.

In some aspects, the techniques described herein relate to an apparatus including: an organ viability analysis instrument including: an in situ organ interrogation module including a positioned photoacoustic array with predetermined spectral and directional laser excitation and acoustic response signal capabilities selected based on an organ type and respective viability factors for an organ in its natural location within a body by performing photoacoustic computational imaging, the photoacoustic array configured to produce one or more first sets of organ response data related to the viability factors for the in situ organ; an ex vivo organ interrogation module including: a stationary photoacoustic array with predetermined spectral and directional laser excitation and acoustic response signal capabilities selected based on organ type and respective viability factors for an ex vivo organ received in an acoustic coupling nest, the photoacoustic array configured to produce one or more second sets of organ response data related to viability factors by performing photoacoustic, multispectral scanning; and one or more direct measurement sensors configured to perform one or more of gravimetric, dimensional, and environmental measurements related to viability factors for ex vivo organ using respective non-wave-based modalities.

### BRIEF DESCRIPTION OF THE DRAWINGS

A more particular description of the examples briefly described will be rendered by reference to specific implementations that are illustrated in the appended drawings. Understanding that these drawings depict only some examples and are not therefore to be considered to be limiting of scope, the examples will be described and explained with additional specificity and detail through the use of the accompanying drawings, in which:

FIG. 1 is a schematic block diagram illustrating an overview of a system for, rapid on-site multiscale multimodal donor organ viability factor characterization, according to one or more examples of the disclosure;

FIG. 2A is an isometric view of an donor organ assessment instrument, according to one or more examples of the disclosure;

FIG. 2B is an alternate isometric drawing of one embodiment of the donor organ assessment instrument configured for travel;

FIG. 2C is a side view drawing of one embodiment of the donor organ assessment instrument configured for travel storage;

FIG. 2D is a schematic block diagram of one embodiment of a main cabinet;

FIG. 3A is a side view drawing of an in-vivo scanning module according to an embodiment;

FIG. 3B is a flow chart diagram of a scanning preparation method according to an embodiment;

FIG. 3C is a flow chart diagram of a scanning method according to an embodiment;

FIG. 3D is a flow chart diagram of a scan switching method according to an embodiment;

FIG. 3F is a schematic diagram of a scanning head according to an embodiment;

FIG. 4A is a schematic diagram of an ex vivo module according to an embodiment;

FIG. 4B is a flow chart diagram of one embodiment of processing an ex vivo organ of interest and preparing it for interrogation;

FIG. 4C is a flow chart diagram of one embodiment of interrogating an ex vivo organ to assess its viability;

FIG. 5A is a schematic block diagram of a hyperspectral hyperspectral biosample analysis station according to an embodiment;

FIG. 5B is a schematic diagram of data collection according to an embodiment;

FIG. 5C is a perspective drawing of organ samples according to an embodiment;

FIG. 5D is a perspective drawing of organ samples according to an alternate embodiment;

FIG. 5E is a side view drawing of organ samples according to an embodiment;

FIG. 5F is a schematic flow chart diagram of a method of imaging an ex vivo organ according to an embodiment;

FIG. 5G is a flow chart diagram of one embodiment of an automated programmatic process;

FIG. 5H is a side view drawing of an organ sample according to an embodiment;

FIG. 5I is a flow chart diagram of classification, segmentation, and visualization process according to an embodiment;

FIG. 5J is a molecular spectroscopy image of an organ according to an embodiment;

FIG. 5K is a graph of spectroscopy results according to an embodiment;

FIG. 5L is an illustration of a hyperspectral biosample according to an embodiment;

FIG. 6A is a schematic block diagram illustrating one embodiment of model data;

FIG. 6B is a schematic block diagram illustrating one alternate embodiment of model data;

FIG. 6C is a schematic block diagram illustrating one embodiment of a computer;

FIG. 6D is a schematic block diagram illustrating one embodiment of a neural network;

FIG. 7A is a flow chart diagram illustrating one embodiment of the diagnostic method; and

FIG. 7B is a flow chart diagram illustrating one embodiment of a diagnostic model training method.

### DETAILED DESCRIPTION

### Disclosure Reading Guidelines

As will be appreciated by one skilled in the art, aspects of the examples may be implemented as a system, apparatus, and or method.

Furthermore, the described features, structures, or characteristics of the embodiments may be combined in any suitable manner. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an example implementation.

As will be appreciated by one skilled in the art, aspects of the disclosure may be implemented as a system, apparatus, method, or program product. Accordingly, aspects or implementations may take the form of an entirely hardware implementation, an entirely software implementation (including firmware, resident software, micro-code, etc.), or an implementation combining software and hardware aspects that may all generally be referred to herein as a “module,” “controller,” or “system.” Furthermore, aspects of the disclosed subject matter may take the form of a program product implemented in one or more computer readable storage devices storing machine-readable code, computer readable code, and/or program code, referred to hereafter as code. The storage devices may be tangible, non-transitory, and/or non-transmission. The storage devices may not embody signals. In a certain implementation, the storage devices only employ signals for accessing code.

Various of the functional units described in this specification have been labeled as modules or controllers. Certain of the modules described in the specification are primarily mechanical and/or fluidic modules. Some functions of a module or a controller may be implemented as a hardware circuit comprising semiconductors such as logic chips, transistors, or other discrete components, or conductors.

For example, one or more modules may include an NFC tag used to convey information about a blood collector module, a plasma separator module, a transfer module, and so forth. A module or controller may also be implemented in programmable hardware devices such as field-programmable gate arrays, programmable array logic, programmable logic devices, or the like.

Certain types of modules or controllers may also be implemented in part or in whole, in code and/or software for execution by various types of processors. An identified controller or module of code may, for instance, comprise one or more physical or logical blocks of executable code which may, for instance, be organized as an object, procedure, or function. Nevertheless, the executables of an identified controller or module need not be physically located together but may comprise disparate instructions stored in different locations which, when joined logically together, comprise the module and achieve the stated purpose for the controller or module.

Indeed, a controller or a module of code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different computer readable storage devices. Where a controller, module, or portions thereof are implemented in software, the software portions are stored on one or more computer readable storage devices.

Any combination of one or more computer readable medium may be utilized. The computer readable medium may be a computer readable storage medium. The computer readable storage medium may be a storage device storing the code. The storage device may be, for example, but not limited to, an electronic, magnetic, optical, electromagnetic, infrared, holographic, micromechanical, or semiconductor system, measurement apparatus, or device, or any suitable combination of the foregoing.

More specific examples (a non-exhaustive list) of the storage device would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, measurement apparatus, or device.

Code for carrying out operations for some implementations may be written in any combination of one or more programming languages including MATLAB, IDL® for multimodal, geospatial, and especially for hyperspectral data visualization and analysis in multiple dimensions an object-oriented programming language such as TensorFlow, R, Python, Ruby, Java, Smalltalk, C++, or the like, and conventional procedural programming languages, such as the “C” programming language, or the like, and/or machine languages such as assembly languages. The code may execute entirely on the subject's computer, partly on the subject's computer, as a stand-alone software package, partly on the subject's computer and partly on a remote computer, or entirely on the remote computer or server. In the latter scenario, the remote computer may be connected to the subject's computer through any type of network, including a local area network (LAN) or a wide area network (WAN), or the connection may be made to an external computer (for example, through the Internet using an Internet Service Provider).

Reference throughout this specification to “one example,” “one implementation,” “an example,” “an implementation,” or similar language means that a particular feature, structure, or characteristic described in connection with the example is included in at least one example. Thus, appearances of the phrases “in one example,” “in an example,” “in an implementation,” and similar language throughout this specification may, but do not necessarily, all refer to the same example or implementation, but mean “one or more but not all examples” unless expressly specified otherwise. The terms “including,” “comprising,” “having,” and variations thereof mean “including but not limited to,” unless expressly specified otherwise. An enumerated listing of items does not imply that any or all of the items are mutually exclusive unless expressly specified otherwise. The terms “a,” “an,” and “the” also refer to “one or more” unless expressly specified otherwise.

As used herein, a list with a conjunction of “and/or” includes any single item in the list or a combination of items in the list. For example, a list of A, B, and/or C includes only A, only B, only C, a combination of A and B, a combination of B and C, a combination of A and C, or a combination of A, B, and C. As used herein, a list using the terminology “one or more of” includes any single item in the list or a combination of items in the list. For example, one or more of A, B, and C includes only A, only B, only C, a combination of A and B, a combination of B and C, a combination of A and C, or a combination of A, B, and C. As used herein, a list using the terminology “one of” includes one and only one of any single item in the list. For example, “one of A, B, and C” includes only A, only B, or only C and excludes combinations of A, B, and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B, and C.

Also, as used herein, the term “about” generally means within +10%, +5%, +1%, or +0.5% of a given value or range, unless otherwise clear from context.

Aspects of the examples and/or implementations are described below regarding schematic flowchart diagrams and/or schematic block diagrams of methods, apparatuses, systems, and program products according to various example implementations.

The flowchart diagrams and/or block diagrams in the Figures illustrate the architecture, functionality, and operation of possible implementations of apparatuses, systems, methods, and program products according to various examples and implementations. In this regard, each block in the flowchart diagrams and/or block diagrams may represent a module, segment, or portion of code, which includes one or more executable instructions of the code for implementing the specified logical function(s).

It should also be noted that, in some alternative implementations, the functions noted in the block may occur out of the order noted in the Figures. For example, two blocks shown in succession may be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Unless otherwise clear from context, like numbers refer to like elements in all figures, including alternate implementations of like elements.

### Definitions

As used herein, the term “biosample” and related terms such as “biosample analysis” may be used to refer to samples collected in relation to organ viability where spectroscopic and other analyses of any biological material derived from a donor organ or associated with the viability assessment of a donor organ may be performed. This includes, but is not limited to: organ biopsy samples, tissue samples, fluid samples, and constituent components and molecules thereof. The analysis may include measuring and comparing molecular components of the organ or other sample substance using hyperspectral imaging and measurement as well as other measurement techniques. For example, biosample analysis may include hyperspectral imaging techniques to capture detailed spectral information across different wavelengths from the biosample. This includes the examination of tissue, blood, and other biological materials to identify specific molecular signatures, chromophore distributions, and other spectral characteristics that are indicative of organ viability.

As used herein, the term “computational photoacoustic imaging” refers to an advanced imaging technique that combines the principles of photoacoustic effect and computational algorithms to produce high-resolution images of biological tissues. In this method, short laser pulses are used to irradiate the tissue, causing rapid thermal expansion and generating ultrasonic waves due to the photoacoustic effect. These ultrasonic waves are then detected by ultrasonic transducers and processed using sophisticated computational algorithms to reconstruct detailed images of the tissue's internal structures. This technique leverages the high contrast provided by optical absorption and the high spatial resolution of ultrasound, making it particularly useful for visualizing vascular structures, tumor margins, and other features that are difficult to discern with traditional imaging modalities.

The computational aspect of photoacoustic imaging involves the use of advanced signal processing and image reconstruction algorithms to enhance image quality and resolution. These algorithms can correct for factors such as acoustic attenuation, scattering, and limited-view artifacts, thereby improving the accuracy and depth of the reconstructed images. Additionally, computational techniques can integrate multi-wavelength photoacoustic data to provide functional and molecular information about the tissue, such as oxygen saturation levels and the presence of specific biomarkers. As a result, computational photoacoustic imaging holds significant promise for non-invasive medical diagnostics, real-time monitoring of therapeutic interventions, and fundamental biological research.

### TECHNOLOGY OVERVIEW AND INTRODUCTION

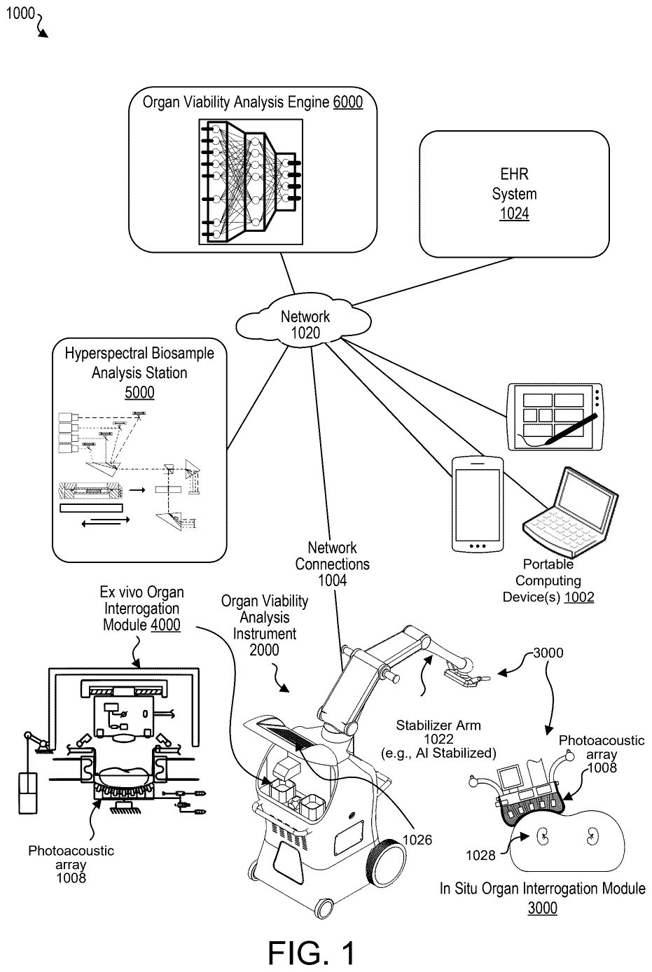

FIG. 1 is a schematic block diagram of an organ viability assessment apparatus 1000, according to one or more examples of the present disclosure. The apparatus 1000, as illustrated in FIG. 1, is designed for the rapid on-site multiscale multimodal characterization of donor organ viability factors. This advanced system integrates various modules and components to provide comprehensive, real-time analysis of donor organs both in situ (within the body) and ex vivo (outside the body).

In various implementations apparatus includes an in situ organ interrogation module (3000) that includes a positionable photoacoustic array 1008 which is supported by a stabilizer arm 1022 and position to direct the laser energy of the photoacoustic array 1008 towards an organ in a body. For example, an in situ organ interrogation module 3000 may allow the photoacoustic array 3000 to make good acoustic contact and facilitate deep tissue excitation This module incorporates a photoacoustic array capable of predetermined spectral and directional laser excitation and acoustic response signal capabilities. It is designed to produce organ response data related to viability factors for an organ in its natural location within the body.

Stabilizer Arm: The module is equipped with a stabilizer arm to couple the photoacoustic array to a specific body region, ensuring stable communication of photoacoustic signals. The arm can electromechanically secure the photoacoustic array based on organ response data to enhance interrogation accuracy.

### Ex Vivo Organ Interrogation Module (4000):

Stationary Photoacoustic Array: This array is configured similarly to the in situ array but is designed for organs that have been removed from the body. It performs photoacoustic multispectral scanning on an organ placed in an acoustic coupling nest.

Acoustic Coupling Nest: The nest provides a stable environment for the ex vivo organ, facilitating accurate measurements.

Direct Measurement Sensors: These sensors perform gravimetric, dimensional, and environmental measurements using non-wave-based modalities to assess organ viability.

Hyperspectral biosample analysis station (5000): In some examples, the Hyperspectral biosample analysis station is implemented using discrete Quantum Cascade Laser Diodes: The analyzer uses discrete frequency laser diodes to perform spectroscopic scanning of biopsy samples, distinguishing pathological organ components from constitutive ones based on the selected organ type.

Controller and Cartridge: The analyzer includes a controller to adjust scanning coordinates and timing, and a cartridge with selectively spectrally transmissive windows for safe spectroscopic scanning.

Organ Viability Analysis Engine (2000): in various implementations the system 1000 includes and organ viability analysis engine 6000, (which may be referred to as simple the “analysis engine” or the “OVAE,”, The analysis engine may perform Signal Processing and be training using Machine Learning to perform organ viability analysis functions. The analysis engine 6000 may incorporate signal preprocessing functions to normalize, filter, and denoise data. Feature extraction and machine learning algorithms analyze these features to generate predictive models assessing organ viability.

Data Integration: The engine integrates data from the in situ and ex vivo modules, as well as the spectral biopsy analyzer, to provide comprehensive organ viability assessments.

Hyperspectral Biologic Sample Analysis Station (1004): In various examples this station supports the organ viability analysis engine 2000, (which may be referred to as simple the “analysis engine” or the “OVAE,” by providing additional hyperspectral data on biological samples, enhancing the overall diagnostic capability of the system.

Portable Computing Devices (1002): These devices facilitate on-site data processing and user interface functionalities, enabling clinicians to operate the apparatus and review results in real-time.

EHR System (1026): The system 1000 may include data integrations to or from one or more electronic health record (EHR) systems 1024 to incorporate patient data, providing context for the organ viability assessments and ensuring data traceability and compliance with health information standards.

In various example, the system 1000 operates by first positioning the in situ organ interrogation module (3000) over the organ (1028) of interest within the body. The photoacoustic array then performs computational imaging, generating detailed viability data. For organs removed from the body, the ex vivo module performs similar analyses with the organ placed in the acoustic coupling nest. The hyperspectral biosample analysis station can further characterize biopsy samples, providing additional data on organ health.

Data from these modules is processed by the organ viability analysis engine, which uses advanced signal processing and machine learning algorithms to generate predictive models. These models assess various viability factors, such as oxygenation, chromophore distribution, and fibrosis status, providing clinicians with detailed insights into the organ's condition.

The system 1000, may include one or more portable computing devices 1002 that enable on-site operation and data review, while integration with one or more EHR systems 1024 ensures comprehensive data management and compliance with health standards.

Network Connections: The system 1000 may include one or more network connections (1004) to facilitate data transfer between the different modules, the analysis engine 6000, and external systems such as the EHR system 1024. This connectivity which may include wireless and/or wired network connections ensures seamless data flow and integration, supporting efficient and accurate organ viability assessments.

By combining advanced photoacoustic technology, hyperspectral analysis, and machine learning, the apparatus 1000 provides a robust solution for rapid and comprehensive donor organ viability characterization, aiding in the equitable allocation and utilization of donor organs. Further details concern the system 1000, the organ viability analysis instrument 2000 and other components depicted in FIG. 1 is provided below in the descriptions corresponding to other Figures.

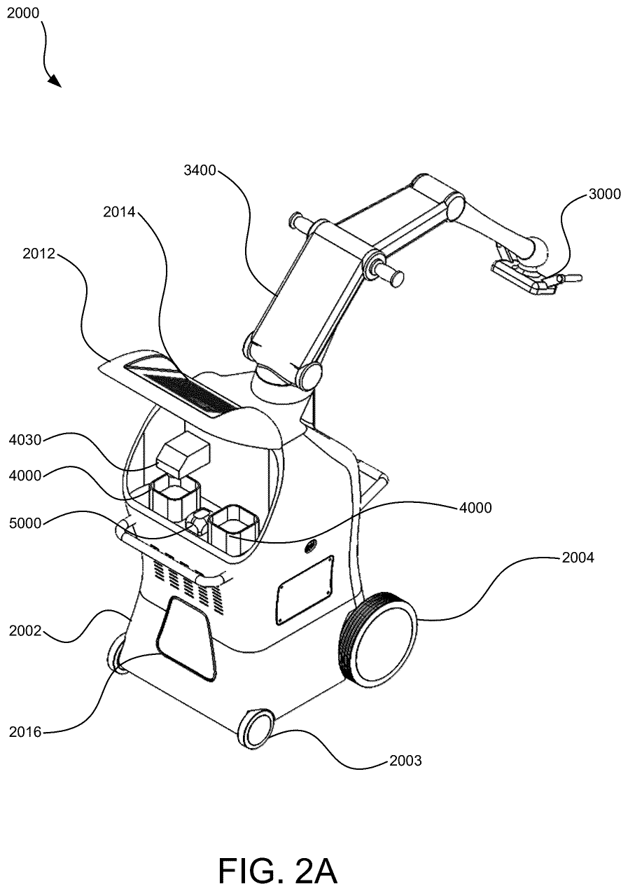

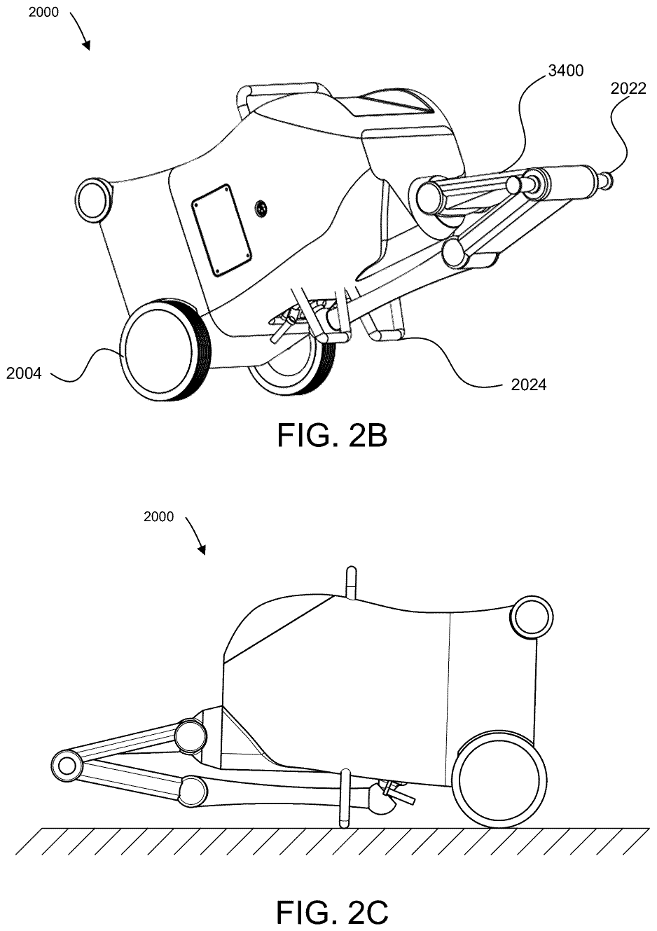

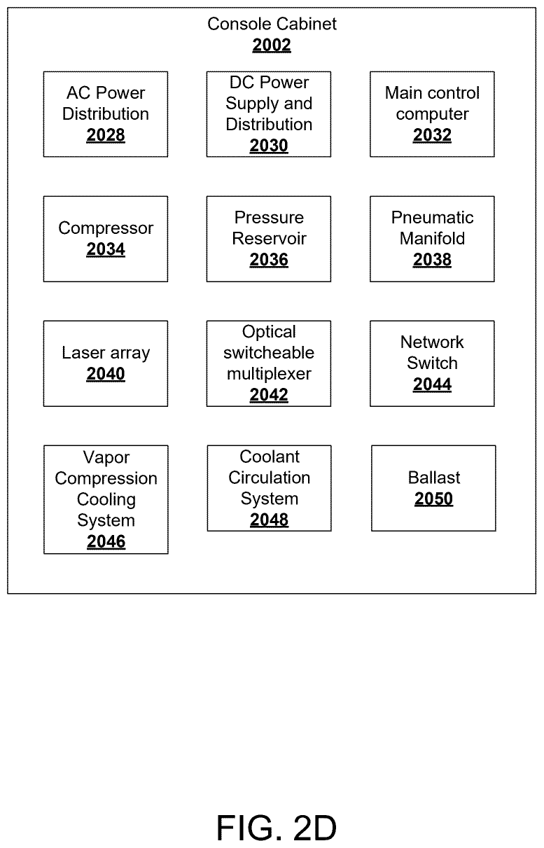

FIG. 2A is an isometric view of the donor organ assessment instrument 1000 in a console that is transportable. The console consists of a main cabinet (2002) on rubberized shock resistant wheels and casters, intended for weather tolerance and suited for travel by air, auto, or any other mode (2004) which allows for ease of movement and protection of the internal componentry from the natural elements. The wheels (2004) allow for easy relocation of the console between operating theaters, hospital units, and geographies. The casters (2004) are equipped with pneumatic, manually, or otherwise actuated brakes to arrest the console in place when scanning. The casters (2004) are also connected to the main cabinet (2002) via a suspension system that ensures floor contact with all wheels/casters. The console 2000 may incorporate one or more ex vivo organ interrogation modules (4000), and or a hyperspectral biosample analysis station (5000). A laser output module on a 3-axis linear motion system incorporates galvanic mirrors to steer the beam illuminates the hyperspectral biosample analysis station (5000).

In some implementations, the modules in this console do not activate their laser analyses unless the operator presses the ‘close shield’ button, followed by the ‘begin ex vivo scan’ or ‘begin hyperspectral biopsy scan’ button. in the case of the ex vivo whole organ photoacoustic module and the hyperspectral biopsy modules; and in the case of the in situ module the lasers do not activate unless the coupling of the transducer array is fully activated and the computerized sensor alert signals this coupling has been achieved (106) that protects operators when the laser output module is active. The console also has a user interface (2014) which may have a touch screen and speakers. It may also incorporate tactile inputs such as a kick pad (2016). The console may also have an arm (3400) that supports an in-vivo scanning module (3000). The arm (3400) has locking joints so to maintain a stable position of the in-vivo scanning module (3000) when scanning.

FIG. 2B depicts the console in a travel configuration. The arm (3400) folds into the travel configuration and locks. The arm incorporates one or more handles (2200) with hand grips that are configured to enable the console to be easily moved around on just the large wheels (2004). The console also includes supports (2024) that can be used to sustain the console unit in a horizontal position while transiting. This stowed position suits other modes of transportation such as air/boat/aerospace as the support bars themselves are intended to secure the unit to floorboard of a transport vehicle with a ratchet strap and hook s to be securely stowed during transport. This allows the console to be stowed and/or transported in a horizontal position, to fit into vertically constrained spaces and or to make it more stable during transportation by lowering its center of gravity.

FIG. 2C depicts the console in a travel stowage configuration. With the arm (3400) folded into the travel configuration and locked, the console can be configured to rest on supports (2024) when stowed during transport. This allows the console to be on its side, to fit into vertically constrained spaces such as for example an automobile, an airplane, a boat, and so forth. A generally horizonal position also makes the console more stable during transportation by lowering its center of gravity.

FIG. 2D is a schematic block diagram of a main cabinet 2002 of the console 2000. Figure depicts the internal systems and components of the console cabinet. The console 2000 accepts AC power and distributes it via AC Power Distribution (2028). The DC power supply and distribution (2030) converts some of the AC power to necessary voltages of DC power and distributes it to other components, such as the main control computer (2032). The main control computer (2032) operates all the console subsystems, runs the user interface, and communicates data to and from the organ analysis engine. The console 2000 also incorporates a compressor (2034), pressurized air reservoir (2036), and pneumatic manifold (2038).

A pneumatic manifold (2038) supplies pressurized air to operate actuators for brakes in wheels and support arm joints (2016), and any other subsystems that need it. An in situ donor organ interrogation module, which is described in more detail below, incorporates an array of laser sources (2034) that output coherent light at wavelengths useful for photoacoustic and/or spectroscopic analysis. The laser array (2040) fires into an optical switchable multiplexer (2042) that routes the light to the appropriate scanning location via a network of fiber optic light pathways. A network switch (2044) routes data between subsystems and the main computer (2032) as well as the organ analysis engine.

The cabinet may also incorporate a vapor compression cooling system (2046) and a coolant circulation system (2048). These will maintain the kidneys in ex vivo modules at acceptable temperature as well as provide temperature control for any other subsystems as necessary. The console main cabinet may incorporate a ballast (2050) to ensure stability.

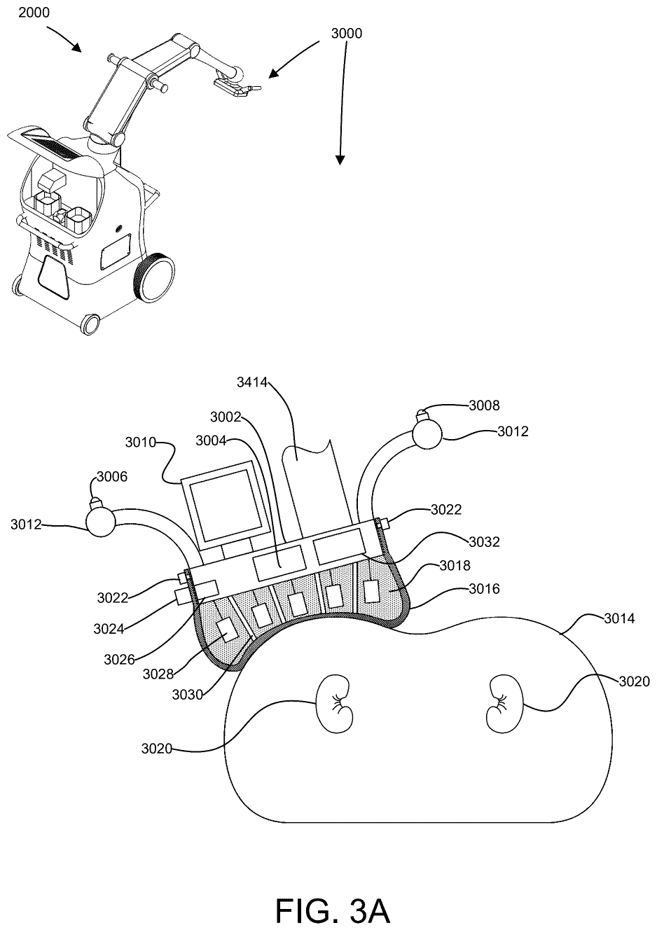

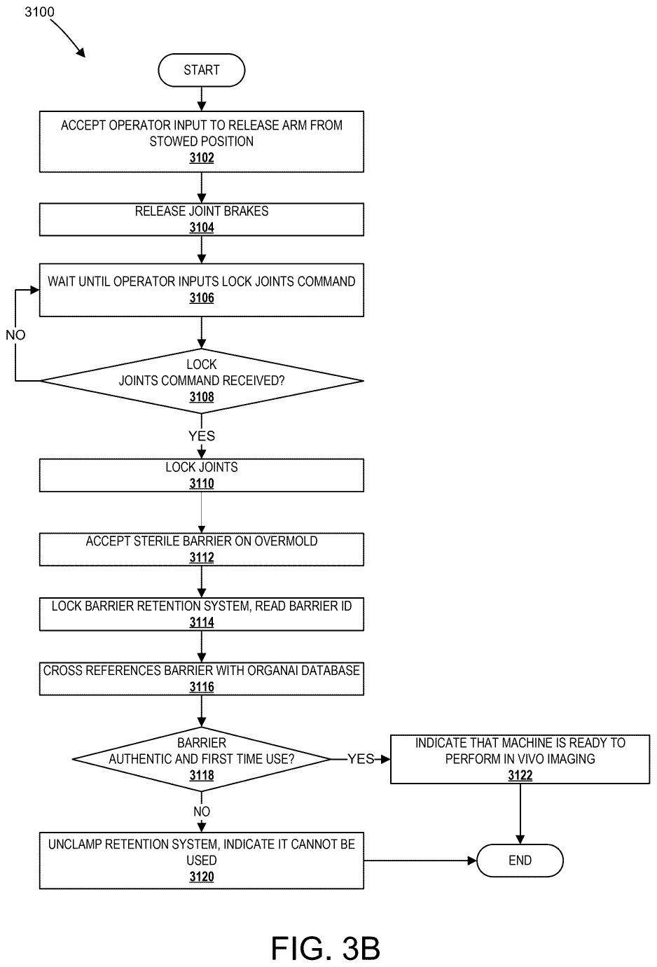

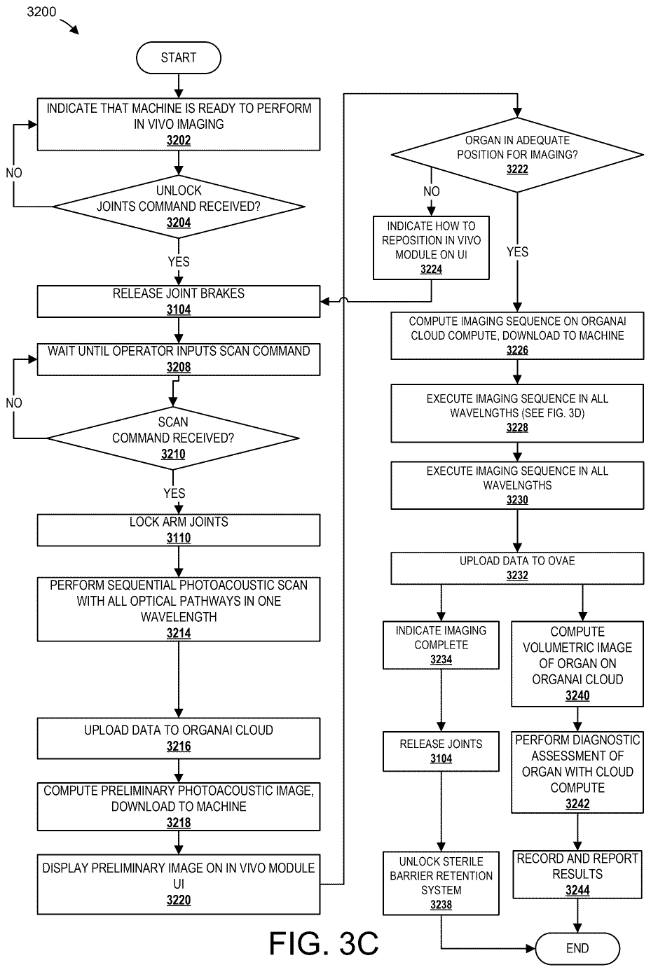

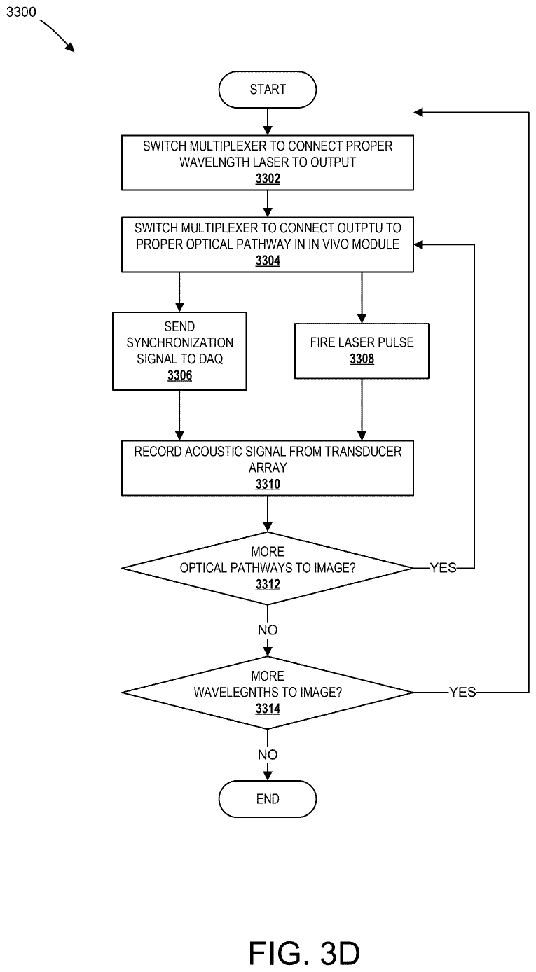

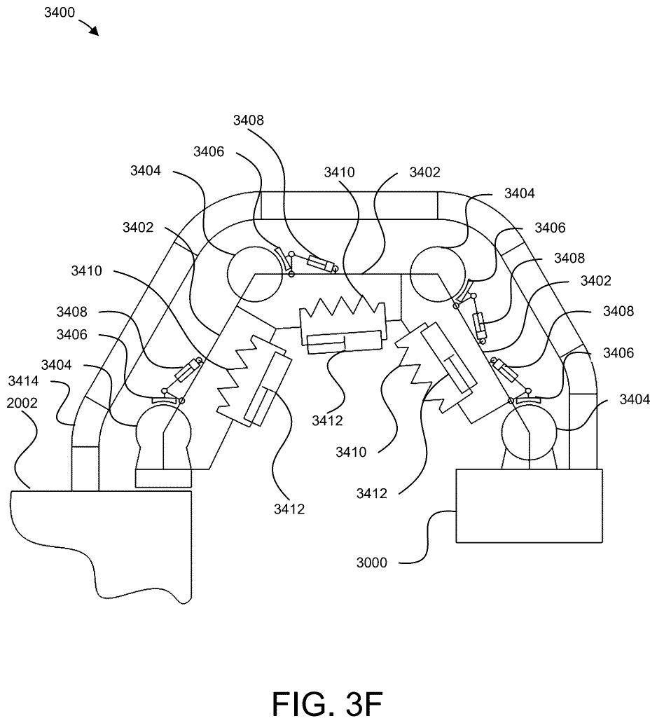

FIG. 3A depicts the in situ organ interrogation module (3000). The in situ organ interrogation module 3003 includes of a rigid frame (3002) that houses a microcontroller (3004) for processing user inputs via an arm unlock button (3006), a scan button (3008), and to operate a small display to relay prompts to the operator. The rigid frame (3002) incorporates handles which the operator uses to position the n situ organ interrogation module (3000) against a patient body (3014) such that the sterile barrier (3016) and phantom gel overmold (3018) are in firm contact, and over one or other kidney (3020). The sterile barrier (3016) prevents patient/machine cross contamination. It attaches to the rigid frame via a retention system (3022). It also incorporates an optical or rf identification system with a unique serial number (3024). The rigid frame (3002) houses a serial number scanner (3026).

The phantom gel overmold (3018) encloses a transducer (3028) array and an optical pathway (3030) array. The phantom gel overmold (3018) and sterile barrier (3016) are elastic and conform to the shape of the body (3014) to facilitate photoacoustic signal transmission.

The rigid frame also houses a data acquisition device (3032) to collect acoustic readings from transducers (3028) and communicate them to the main control computer (3400). All connections to the console cabinet (2002) are via the conduit system (3414) incorporated into the support arm.

During imaging main control computer (3400) fires appropriate lasers in the photoacoustic array (1006,1008) and routes them to proper optical pathways (3030) via the optical switchable multiplexer (2042). The data acquisition device then reads transducer (3028) signals and sends the data back to the main control computer. When the scan is done, the main control computer (3400) indicates to the operator via small display to either position the in-vivo module over the other kidney (3020) or that it's done.

In situ Module Data. Photoacoustic technology is employed using both transmission and reflection modes ultrasound localizes the target of interest and molecular chemistry enabled acoustic images inform pathological status according to key tissue specific chromophores such as hemoglobin (deoxygenated and oxygenated), collagen, with hemoglobin acting as an endogenous contrast agent content and. Optimal wavelength selection is the first step to quantifying collagen (the primary protein in fibrosis) in the presence of oxyhemoglobin and deoxyhemoglobin (the main light-absorbing molecules in most biological tissues). This step begins with selection of optimal wavelengths to ensure stable spectral unmixing solutions. Optimal wavelengths can be chosen using methods such as those that incorporate ‘extinction’. One such method incorporates variance inflation factor (VIF) derived from the extinction coefficients of key chromophores—in this case collagen, oxyhemoglobin, and deoxyhemoglobin. Photoacoustic computed tomography (PACT) has advanced utility in clinical diagnostics. This is largely due to its ability to balance high resolution, deep penetration, and sensitivity to functional and molecular contrasts. In PACT, a laser excites target generating ultrasound waves through the photoacoustic effect. These waves are detected by an ultrasonic transducer array, which then reconstructs the original optical energy distribution within the targets. Optical absorption contrast images are digitally created using inverse algorithms, allowing for detailed imaging of various functional and molecular properties of biological tissues, such as blood oxygenation, tissue temperature, and molecular probe distribution, typically with multispectral excitation.

In PACT, light is spread out to illuminate the entire target. Photoacoustic signals are collected from multiple locations around the region of interest with the use of the transducer array. Generating Photoacoustic Signals: In PACT, pulsed laser up the target tissue, causing it to absorb light and emit ultrasound waves, and such waves that are then picked up by sensors. Creating the Initial Image: The sensors use the ultrasound signals to make an initial image of the tissue. This image might be blurry or lack detail due to various hardware limitations and wave scattering. Deconvolution Process: Deconvolution is a mathematical method to reduce blurring and improve image quality. Methods of deconvolution can include Point Spread Function (PSF): Understanding how a single point of photoacoustic waves spreads out when detected, which shows the blurring effect. And with Mathematical Correction signals are processed to fix distortions and blurring, Enhanced Image: The result is a clearer, sharper image with better resolution and contrast, allowing for more accurate analysis of the tissue's properties. Deconvolution in PACT is a key step that improves the initial blurry image, making it clearer and more detailed for better analysis of organ's structural characteristics. Important to our image processing technique are Low-frequency PA signals (<1 MHz). This signal range is important for PACT despite usually being overlooked due to typically manifesting as low resolution during standard image reconstruction techniques. To mitigate the effect of PACT reconstruction methods which typically utilize filters that eliminate low frequency signals. Instead we employ deconvolution with the use of a filter such as Wiener or Tikhonov to preserve important low frequencies.

Two processes drive OA/PA signal acquisition. First, the optical forward process describes the generation of initial pressure derived from chromophore concentrations and the light distribution (fluence) within the 3-D medium. Second, the acoustic forward process describes the acquisition of acoustic waves originating from the initial pressure.

Oxygen saturation (SO2) levels are crucial to renal functional assessment with normal SO2 typically ranges between 70-75% and the oxygen saturation in renal venous blood (reflecting the oxygen extraction by the kidney) is under normal conditions, indicating a healthy balance between oxygen supply and demand. Alternatively, hypoxia or restricted oxygen saturation SO2 levels pointing to renal impairment are less than 60-65% suggestive of signs of renal distress or dysfunction. Normothermic regional perfusion (NRP) is a relatively new practice in organ donation, aimed at mitigating risk of warm ischemic time by artificially sustaining organs of a deceased donor following death declaration while consent from families and other pre-procurement protocol are carried out.

Clinical consensus currently exists regarding the NRP and a need for pre-post data to guide the timepoint thresholds for each unique organ when damage despite the imposed perfusion of NRP begins to set in. This need for data to guide the practice and establish NRP standards is especially important for organs that are less hardy than the kidney which has up to 30 hours of viability post-procurement. For organs such as the heart (4-6 hours) and lungs (4-8 hours) the crossover when risks posed by warm ischemia, progresses to imminent organ damage, is characterized as a tenuous tipping point. Tech advancements for clinical decision support tools to mitigate ambiguity in organ refusal (discard) decisions to refuse (discard) on the basis of non-data driven risk aversion/avoidance. Deceased donors following brain death can have widely varied SO2 levels for any number of reasons, making rapid precise measurements of organ perfusion or altered or wide fluctuations in oxygen demands have key implications for organ viability status assessments at the point of care. Signs of vascular injury may include localized reductions in perfusion, changes in the pattern of perfusion indicative of disruption in blood flow, or abnormal SO2 levels. OA/PA is the ideal technology to identify these regions by monitoring and highlighting significant changes in blood volume and oxygenation and ascertaining likelihood of damage incurred to maximize the rate of deceased donor transplants. The ultimate goal of OA/PA imaging is to accurately quantify chromophore concentrations from acquired data. In general, two steps are required to solve this inverse problem. (1) initial pressure distribution is reconstructed by addressing the acoustic inverse problem. Then, chromophore concentrations are estimated by solving the optical inverse problem using the pressure map as input. The kidneys are composed of two dominant chromophores that contribute to the PA signal: blood (in the form of oxygenated and deoxygenated hemoglobin) and collagen (the core component of fibrosis).

The donor organ viability analysis engine may be configured to use various pre-processing and signal processing functions to process these data to generate a multimode spectral map or a volumetric map of molecules of interest such as oxygenated and deoxygenated hemoglobin, and collagen. Once that is complete, machine learning and or artificial intelligence functions or ensembles may be configured to derive scoring algorithms to assess the organ for transplant viability or perform other diagnostic functions and report back to the clinician.

FIG. 3B is a flow chart diagram of a scanning preparation method 3100 for preparing the imaging arm for in vivo imaging, according to one or more examples of the disclosure. The method involves a series of steps to ensure the imaging arm is correctly positioned and ready for safe operation.

Starting with the initiation of the method, in some implementations, the method 3100 includes accepting (3102) operator input to release the arm from its stowed position. This step initiates the preparation process. In various implementations, the method 3100 includes releasing (3104) the joint brakes to allow movement of the arm.

In certain implementations, the method 3100 includes waiting (3106) until the operator inputs the lock joints command. This ensures that the arm is positioned correctly before locking. In one or more implementations, the method 3100 includes checking (3108) if the lock joints command has been received. If no command is received, the process waits. If the command is received, in some implementations, the method 3100 includes locking (3110) the joints to secure the arm in the desired position.

In various implementations, the method 3100 includes accepting (3112) a sterile barrier on the overmold to maintain a sterile environment during imaging. In certain implementations, the method 3100 includes locking (3114) the barrier retention system and reading the barrier ID to ensure the barrier is properly secured and identified.

In one or more implementations, the method 3100 includes cross-referencing (3116) the barrier with the OrganAI database to verify its authenticity and suitability for use. In some implementations, the method 3100 includes checking (3118) if the barrier is authentic and being used for the first time. If the barrier is not authentic or has been used before, the process moves to unclamping (3120) the retention system and indicating that the barrier cannot be used.

If the barrier is authentic and unused, in various implementations, the method 3100 includes indicating (3122) that the machine is ready to perform in vivo imaging. This step signals that the preparation process is complete and the imaging arm is ready for operation.

This method ensures that the imaging arm is prepared under strictly controlled conditions, maintaining its integrity and readiness for accurate and safe in vivo imaging.

FIG. 3C is a flow chart diagram of a scanning method 3200 for performing in vivo imaging, according to one or more examples of the disclosure. The method involves a series of steps to ensure accurate and comprehensive imaging of an organ in vivo.

Starting with the initiation of the method, in some implementations, the method 3200 includes indicating (3202) that the machine is ready to perform in vivo imaging. This ensures that the system is prepared for the imaging process. In various implementations, the method 3200 includes checking (3204) if the unlock joints command has been received. If the command is received, the method proceeds by releasing (3104) the joint brakes to allow movement of the imaging arm.

In certain implementations, the method 3200 includes waiting (3208) until the operator inputs the scan command. This ensures the scan starts only when the operator is ready. In one or more implementations, the method 3200 includes checking (3210) if the scan command has been received. If the command is received, the method proceeds by locking (3110) the arm joints to secure the imaging arm in place.

In some implementations, the method 3200 includes performing (3214) a sequential photoacoustic scan with all optical pathways in one wavelength. This initial scan collects essential data. In various implementations, the method 3200 includes uploading (3216) the data to the organ viability analysis engine (6000) for processing.

In certain implementations, the method 3200 includes computing (3218) a preliminary photoacoustic image and downloading it to the machine. This step provides an initial visual representation of the organ. In one or more implementations, the method 3200 includes displaying (3220) the preliminary image on the in vivo module UI to inform the operator of the current status.

In some implementations, the method 3200 includes checking (3222) if the organ is in an adequate position for imaging. If the organ is not in the correct position, the method includes indicating (3224) how to reposition the in vivo module on the UI. If the organ is correctly positioned, the method includes computing (3226) the imaging sequence on the organ viability analysis engine (6000) and downloading it to the machine.

In various implementations, the method 3200 includes executing (3228) the imaging sequence in all wavelengths. This comprehensive scan gathers detailed data across multiple wavelengths. In certain implementations, the method 3200 includes uploading (3232) the data to the organ viability analysis engine (6000) for further analysis.

In one or more implementations, the method 3200 includes indicating (3234) that imaging is complete. Following this, the method includes releasing (3104) the joints to allow repositioning of the imaging arm. In some implementations, the method 3200 includes computing (3240) a volumetric image of the organ on the organ viability analysis engine (6000) providing a detailed three-dimensional representation.

In various implementations, the method 3200 includes performing (3242) a diagnostic assessment of the organ with the organ viability analysis engine (6000). This step analyzes the imaging data for diagnostic purposes. In certain implementations, the method 3200 includes recording and reporting (3244) the results, providing a comprehensive report of the imaging session.

Finally, in one or more implementations, the method 3200 includes unlocking (3238) the sterile barrier retention system and indicating that the imaging process is complete, concluding the method.

This method ensures that in vivo imaging is performed accurately and comprehensively, maintaining the integrity of the organ and providing detailed data for diagnostic and medical purposes.

FIG. 3D is a flow chart diagram of a scan switching method (3000) for sequential photoacoustic imaging, according to one or more examples of the disclosure. The method involves a series of steps to perform imaging using different wavelengths and optical pathways to gather comprehensive data.

Starting with the initiation of the method, in some implementations, the method 3300 includes switching (3302) the multiplexer to connect the proper wavelength laser to the output. This step ensures the correct laser wavelength is selected for the imaging process. In various implementations, the method 3300 includes switching (3304) the multiplexer to connect the output to the proper optical pathway in the in vivo module, aligning the laser for accurate imaging.

In certain implementations, the method 3300 includes sending (3306) a synchronization signal to the DAQ (Data Acquisition System). This synchronization ensures that the data collection process is accurately timed with the laser pulses. In one or more implementations, the method 3300 includes firing (3308) the laser pulse to initiate the imaging process.

In some implementations, the method 3300 includes recording (3310) the acoustic signal from the transducer array. This signal provides the data necessary for creating detailed images. In various implementations, the method 3300 includes checking (3312) if there are more optical pathways to image. If there are more pathways, the process loops back to switch the multiplexer to the next optical pathway.

In certain implementations, the method 3300 includes checking (3314) if there are more wavelengths to image. If there are more wavelengths, the process loops back to switch the multiplexer to the next wavelength laser. If no more wavelengths or optical pathways need imaging, the method concludes.

This method ensures that sequential photoacoustic imaging is performed accurately across multiple wavelengths and optical pathways, providing comprehensive data for detailed analysis and imaging.

FIG. 3F is a schematic diagram of a stabilizer arm 3000. The support arm (3004) provides a mechanical load path between the console cabinet (2002) and the in-vivo module (3000). The support arm consists of rigid structural struts (3002 connected by rotating joints (3404) that give the in-vivo module (3000) 6 degrees of freedom when unlocked. This support arm (3004) echolocates the organ beneath the skin recognizing the shape and position of the organ of interest relative to other internal structures and organs, (notifying the operator of the arm's correct positioning of the transducer array with an indicator light and the shape of the organ appearing on the console screen as a full organ silhouette as well. The rotating joints (3404) are equipped with brakes (3406) that lock joint rotation and thus keep the arm fixed while in use or during stow or transport. Pneumatic or some other type of actuator (3408) operates the joint brakes (3406). The main control computer (2032) operates the actuators based on user inputs. Joint springs (3410) keep the support arm from dropping and counteract the weight of the in-vivo module (3000). Dampers (3412) linked in parallel with the springs (3410) prevent excessively rapid joint rotation and thus keep the support arm (3004) from moving too quickly, this reduces chances of impact due to sudden movement. The support arm also features a conduit system (3414) for electrical, pneumatic, optical, and data transmission paths. The conduit system (3414) keeps these transmission paths from being pinched, provides strain relief, and maintains minimum bend radii appropriate for each type of transmission path.

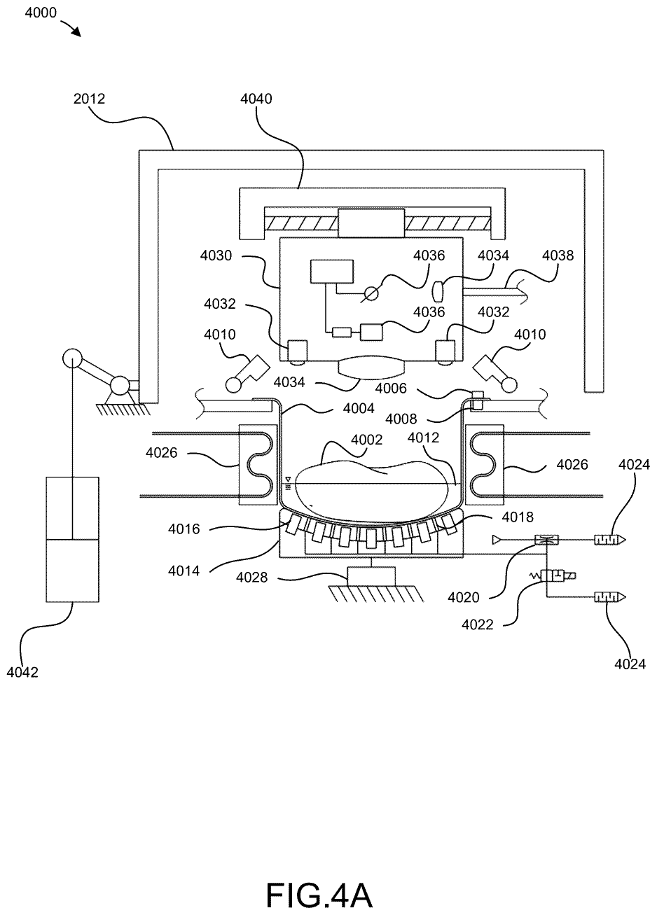

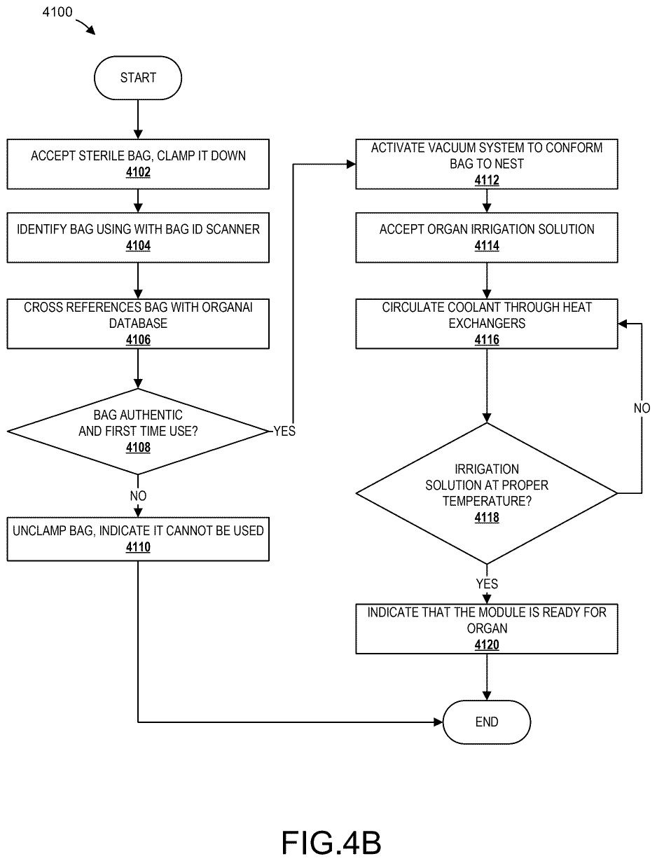

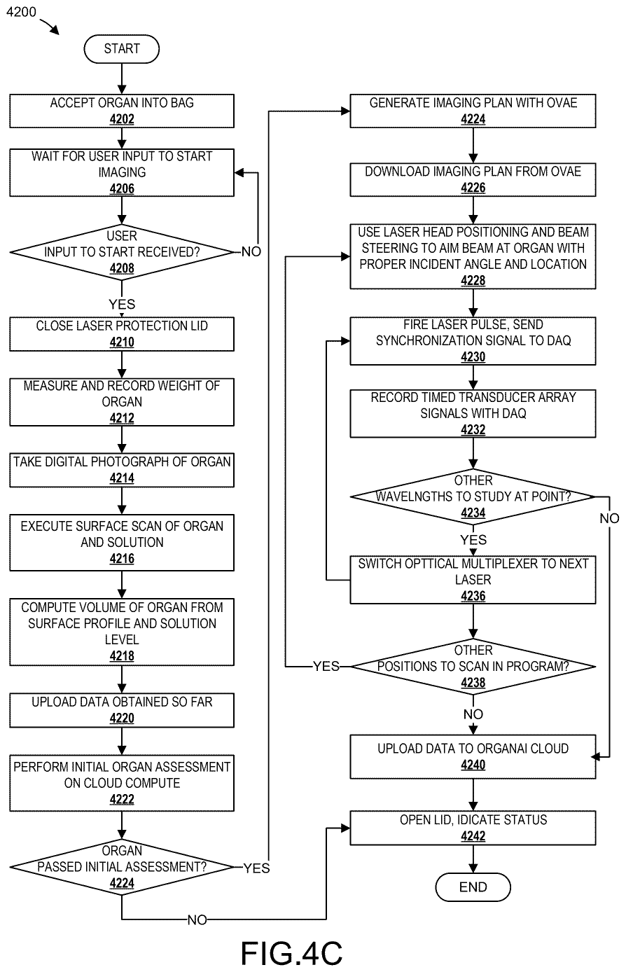

FIG. 4A is an ex vivo module configured to accepts an organ such as a kidney (4002), which the operator inserts into a sterile disposable bag (4004). The bag incorporates an identification tag (4006) that may be optical or rf or some other means. The module in turn incorporates a means to read the tag (4008) creating an automated link via donor id in DonorNet for traceability and health information transfer between deceased donor organ data such as warm ischemic time, blood type, tissue type and then adding to these relevant datapoints donor organ viability screening reports from the OrganAI™ system.

The bag (4004) prevents contamination of the kidney (4002) by the machine and vice versa. The module also has a mechanical locking mechanism for the bag (4010) that seals it to the module. The kidney (4002) is partially submerged in a solution that keeps it from dehydrating (4012). The bag rests on top of an acoustic coupling foam nest (2042) that overmolds a transducer array (4014). The transducers may be based on piezoelectric materials or some other means of converting vibration into electronic signals.

The nest (4016) also incorporates airways (4044) connected to a vacuum generator (4020) and vent valve (4044). One source of error in acoustic measurements is interfaces between dissimilar speed of sound materials, such as air pockets. The combination of airways, vacuum generator, and vent valve (4044) eliminate air pockets between the nest and the bag during operation then release it when it's time to extract the bag, thus mitigating a potential source of inaccuracy. Air from the vacuum generator or vent valve leaves the console via mufflers to mitigate noise from system operation. Heat exchangers use coolant from the coolant circulation system (2048) to keep the kidney (4002) at appropriate temperature. A force sensor, such as a load cell, measures the weight of the organ (4002).

The laser head moves between the two ex-vivo scan modules (3000) to provide illumination and camera views. The laser head incorporates visible and infrared spectrum cameras, laser focus optics, and a beam steering system that uses mirrors on galvanic or other actuators. A fiberoptic light path (4018) connects the head to the optical switch (2042). Linear actuators (4040) move the head in three linear axes to position it over the target (4002) and keep it in the focal plane of the optics. In aggregate the elements of the laser head can scan the shape of the kidney by rastering a laser beam over the kidney (4002) and recording the resulting profile with the cameras. The laser beam can highlight the location of the solution (4012) surface the same way. Thus, if the volume of the solution is known, the surface level of the solution (4012) plus the shape of the kidney (4002) that is above the surface of the solution (4012) will yield the volume of the kidney. Laser beams of appropriate wavelengths will induce photo-acoustic effects in the kidney's (4002) chromophore molecules, and the transducer (4014) array will capture the resulting acoustic signals. The console will transmit these signals to the organ analysis engine which will then compute a volumetric map of chromophore distribution and provide diagnostic scores for the kidney (4002) based on that as well as volume, shape, and weight.

FIG. 4B illustrates a method 4100 for processing an ex vivo organ of interest and preparing it for interrogation. The method involves several steps to ensure the organ is correctly positioned, authenticated, and maintained under optimal conditions for accurate viability assessment.

Starting with the initiation of the method (4100), in some implementations, the method 4100 includes accepting (4102) and clamping (4103) down a sterile bag to secure it in place. This bag will contain the ex vivo organ during the interrogation process. In various implementations, the method 4100 includes identifying (4104) the bag using a Bag ID scanner to ensure that the bag being used is correctly logged and tracked within the system. In certain implementations, the method 4100 includes cross-referencing (4106) the bag ID with the OrganAI database to verify its authenticity and ensure it is appropriate for use with the specific organ being processed.

In one or more implementations, the method 4100 includes performing (4108) a check to determine if the bag is authentic and if it is being used for the first time. This step prevents reuse of bags, which could compromise sterility and accuracy. If the bag fails the authenticity or first-time use check, in various implementations, the method 4100 includes unclamping (4110) the bag and indicating that it cannot be used. The process is halted until a suitable bag is provided. If the bag is authentic and unused, the process continues.

In some implementations, the method 4100 includes activating (4112) the vacuum system to conform the bag to the acoustic coupling nest. This ensures the bag fits snugly around the organ, eliminating air pockets that could interfere with measurements. In certain implementations, the method 4100 includes accepting (4114) organ irrigation solution into the system to help maintain the organ's viability by providing necessary nutrients and maintaining hydration. In various implementations, the method 4100 includes circulating (4116) coolant through heat exchangers to control the temperature of the irrigation solution and the organ. This step is critical to maintaining the organ at a proper temperature throughout the interrogation process.

In some implementations, the method 4100 includes performing (4118) a check to ensure the irrigation solution is at the proper temperature. If the solution is not at the proper temperature, the process loops back to continue circulating coolant. If the solution is at the proper temperature, in one or more implementations, the method 4100 includes indicating (4120) that the module is ready for the organ. The organ can now be placed in the bag and prepared for further interrogation. The process concludes with the organ correctly positioned and maintained within the system, ready for detailed interrogation and analysis.

Accordingly, in various implementations, the method 4100 ensures that the ex vivo organ is handled under strictly controlled conditions, maintaining its viability and integrity for accurate assessment using the interrogation modules of the apparatus.

FIG. 4C illustrates a method 4200 for interrogating an ex vivo organ to assess its viability. The method 4200 involves a series of steps to prepare the organ for detailed examination and analysis.

Starting with the initiation of the method, in some implementations, the method 4200 includes accepting (4202) the organ into the bag. This ensures the organ is securely placed for the interrogation process. In various implementations, the method 4200 includes waiting (4206) for user input to start imaging, ensuring that the process begins only when the operator is ready.

In certain implementations, the method 4200 includes checking (4208) if the user input to start has been received. If no input is received, the process waits. If input is received, in one or more implementations, the method 4200 includes closing (4210) the laser protection lid to ensure safety during imaging.

In some implementations, the method 4200 includes measuring and recording (4212) the weight of the organ. This data is essential for subsequent analysis. In various implementations, the method 4200 includes taking (4214) a digital photograph of the organ to document its initial condition.

In certain implementations, the method 4200 includes executing (4216) a surface scan of the organ and solution. This scan provides detailed surface profile data. In one or more implementations, the method 4200 includes computing (4218) the volume of the organ from the surface profile and solution level.

In some implementations, the method 4200 includes uploading (4220) the data obtained so far. This ensures that all collected data is stored securely. In various implementations, the method 4200 includes performing (4222) an initial organ assessment on the organ viability analysis engine 6000 to analyze the preliminary data.

In certain implementations, the method 4200 includes checking (4224) if the organ passed the initial assessment. If the organ does not pass, the process ends. If the organ passes, in one or more implementations, the method 4200 includes generating (4224) an imaging plan with the organ viability analysis engine 6000.

In some implementations, the method 4200 includes downloading (4226) the imaging plan from the organ viability analysis engine 6000. This plan guides the detailed imaging process. In various implementations, the method 4200 includes using (4228) laser head positioning and beam steering to aim the beam at the organ with the proper incident angle and location.

In certain implementations, the method 4200 includes firing (4230) the laser pulse and sending the synchronization signal to the DAQ. This step initiates the detailed imaging. In one or more implementations, the method 4200 includes recording (4232) timed transducer array signals with the DAQ.

In some implementations, the method 4200 includes checking (4234) if other wavelengths need to be studied at the current point. If yes, in various implementations, the method 4200 includes switching (4236) the optical multiplexer to the next laser.

In certain implementations, the method 4200 includes checking (4238) if there are other positions to scan in the program. If yes, the process loops back to re-position the laser head. If no, in one or more implementations, the method 4200 includes uploading (4240) the data to the for comprehensive analysis.

In some implementations, the method 4200 includes opening (4242) the lid and indicating the status, concluding the interrogation process. This method ensures that the ex vivo organ is thoroughly and accurately assessed, maintaining its viability and integrity throughout the interrogation process, and providing comprehensive data for informed medical decisions.

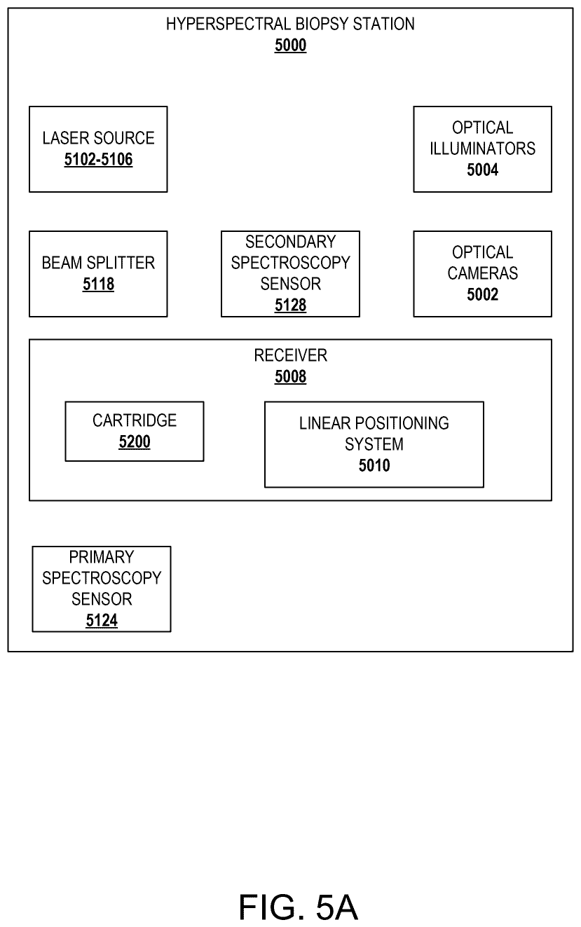

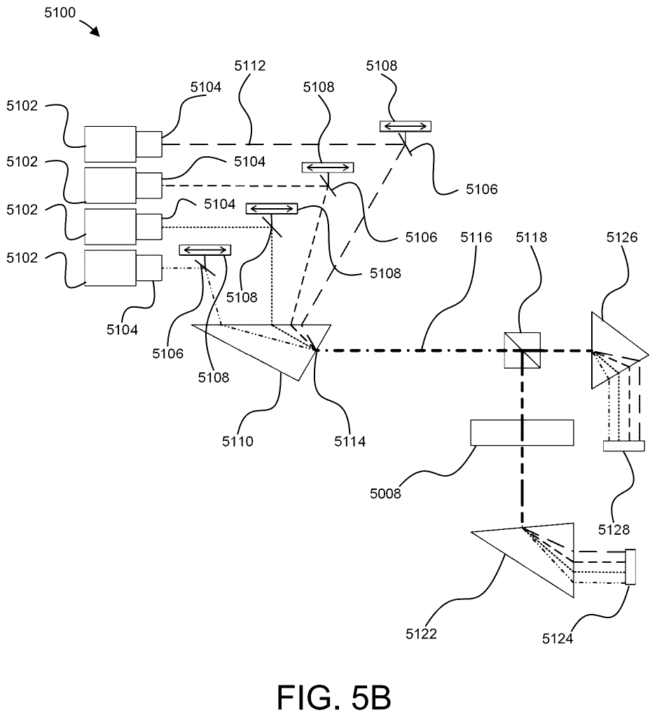

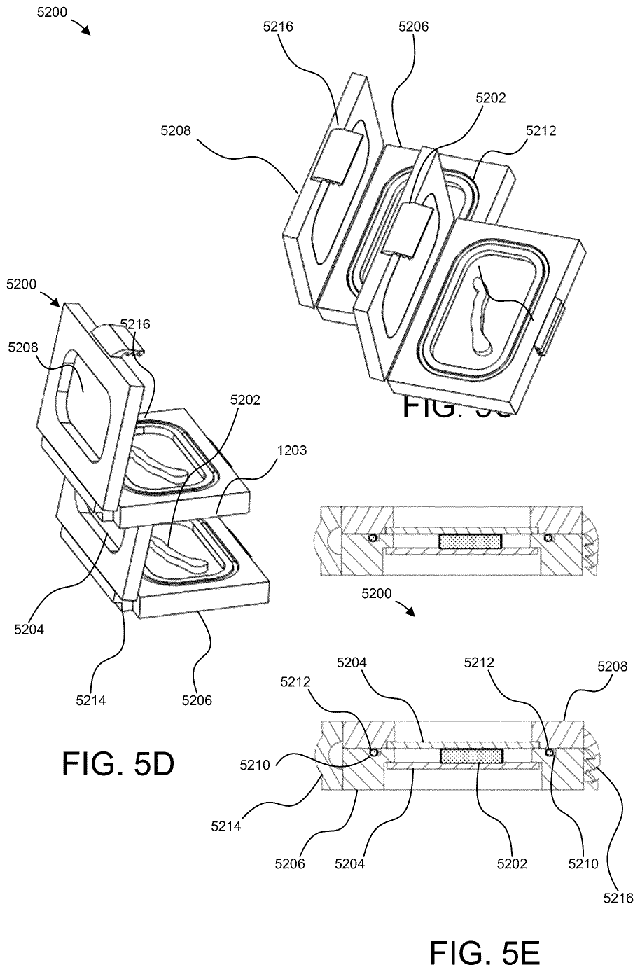

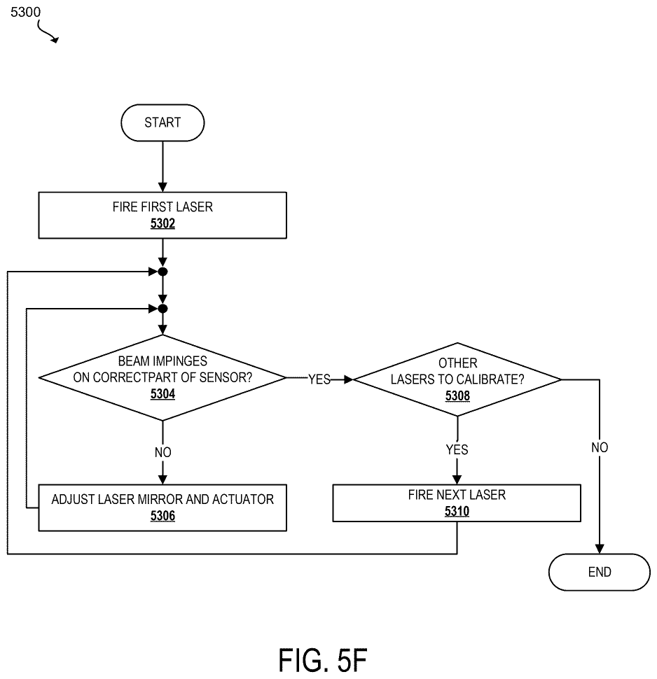

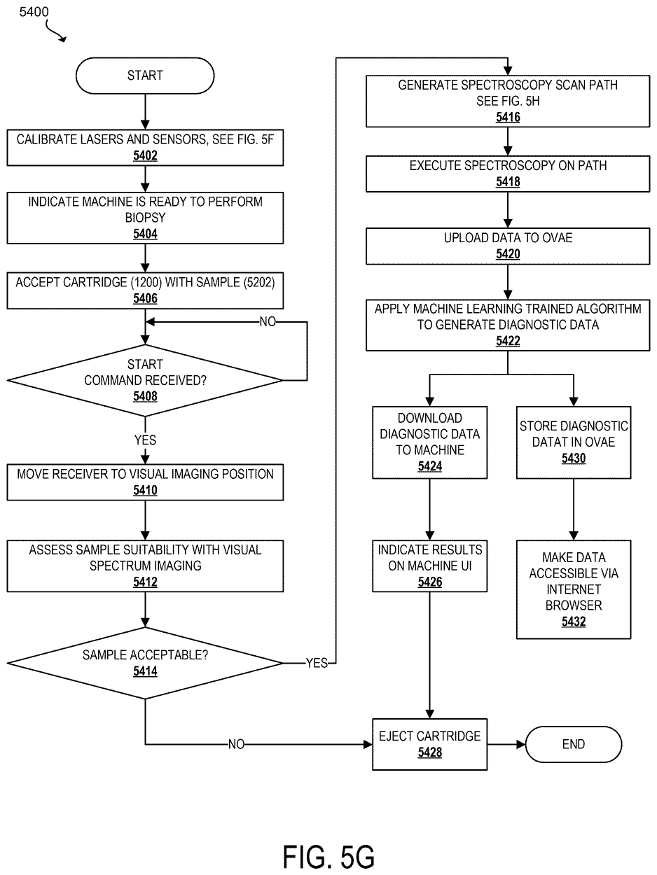

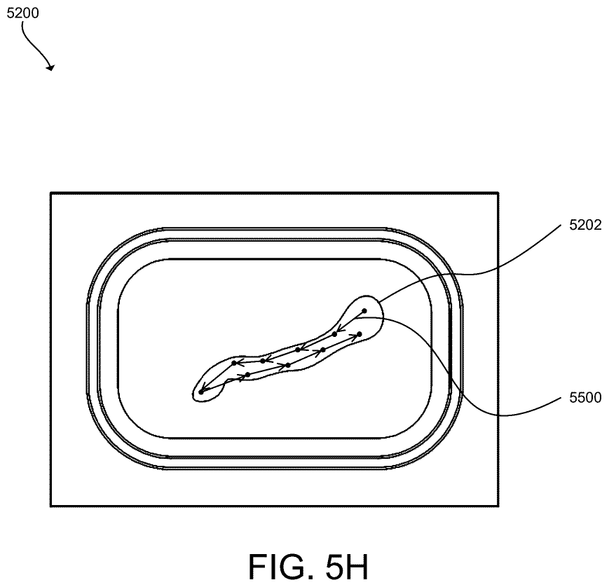

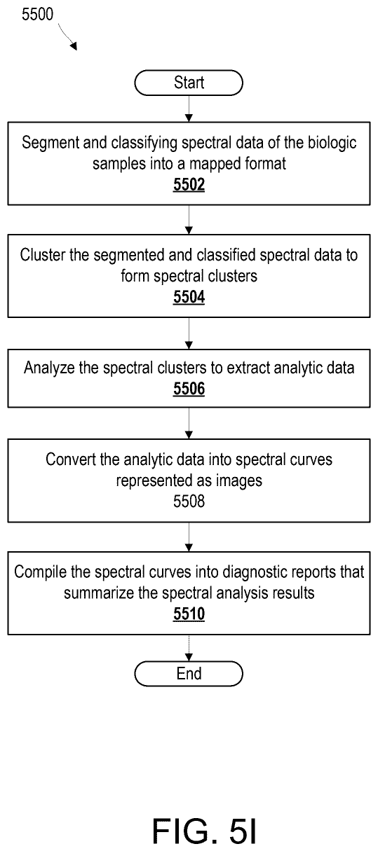

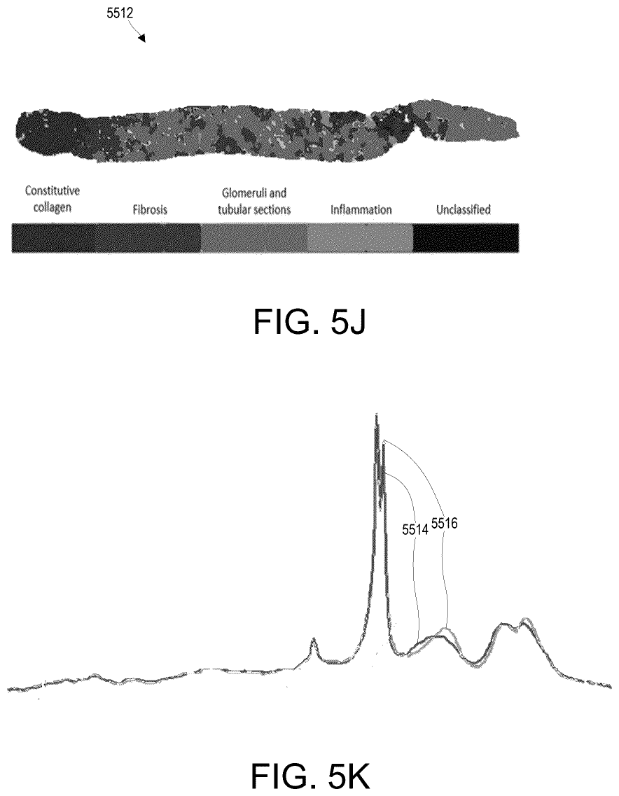

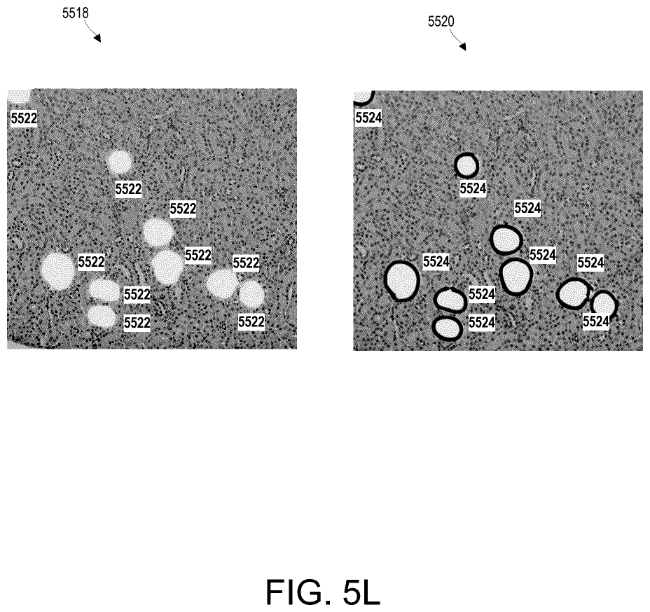

FIG. 5A is a schematic block diagram of a hyperspectral hyperspectral biosample analysis station 5000. depicts a the Lab-Free Hyperspectral biosample analysis station which will employ a discrete frequency (DF) quantum cascade laser (QCL) mid-infrared setup that incorporates a either a mini pulsed laser diode (such as one from Block Engineering Southborough, MA) or a continuous wave (CW) QCL mid-IR laser diode (such as Hamamatsu Photonics, Herrsching am Ammersee,

Germany) operated by a ultrafast driver (option from Meerstetter Engineering GmbH, Rubigen, Switzerland)

to create a multispectral-wavelength laser source diode arrangement (5102) that fires through a beam splitter (5118) and a receiver/mercury cadmium telluride detector (MCT) that operates at room temperature (such as Amplified MCT from DRS Daylight Solutions Inc. San Diego, CA) containing a cartridge (5008) with a biopsy sample in order to perform spectroscopic automated analysis lab-free molecular spectroscopy on fresh, unstained, unlabeled tissue. One output of the beam splitter (5118) fires through the receiver/detector and sample, into the primary spectroscopy light sensor (5124) while the other output fires into the secondary spectroscopy light sensor (5128). The organ viability analysis engine 6000 utilizes response signals from the two light sensors during absorption spectroscopy scans. The hyperspectral biosample analysis station also encompasses optical cameras (5002) that take photographic images of the sample (5202) and read the cartridge (5200) serial number. The main control computer (2032) automatically determines the length, color, pattern analyses the shape of the sample (5202) encapsulated in the cartridge (5200) and plots a path for the multispectral wavelength laser beam to scan the sample, as the series of arrows in FIG. 11 show.

The receiver (5008) incorporates a 2 degree of freedom linear position system (1310) that moves the sample along the computed path during spectroscopy scans. The analytical output of the hyperspectral biosample analysis station can thus not only have bulk absorption spectroscopy scores for the samples, but also localized scores that can be plotted over the optical image of the sample.

Infrared spectroscopy (especially mid-infrared) which leverages the “molecular fingerprint region” is a label-free, stain-free chemical imaging technique that is offering non-destructive, precise, point of care diagnostics tool to vastly improve and automate biopsy, which has remained relatively unchanged since its genesis over a century ago. Discrete frequency mode mid-infrared quantum cascade laser driven spectroscopy is extremely suited to define a new era in automated pathology given its exponentially faster processing, small envelope, room cooled operation, minimal eye risk

The novelty of this invention as a tri-module system extends well beyond each module's precision diagnostic technology, relevant data outputs, efficiencies, and even beyond the physics driven computational methods that advance medicine toward practical and scientifically responsible automation. The most novel aspect of this system is that it benefits from the only domain in medicine that enables a full capture, holistic understanding of all eight human organs immediately at the point of excision and, delivered in-hand. The tri-modal system is a machine built to learn as “its school” is built-in thereby creating a self-sustaining, self-improving diagnostic system that gets smarter over time. from a deeply unique The system is designed to capture of the organ processes and progression tied to regression leading to damage organ deterioration and likewise pinpointing or indictors of organ robustness of organ preservation or even revitalization via imposed machine perfusion.

The infrared spectroscopy capabilities of the delivers chemometric spectral intelligence that informs the optoacoustic applications and vice versa. Consider the deep dive data protocol of a diagnostic system that leverages disease specific, tissue specific, personalized precision diagnostics profiled with clinical contextualized by EHR-pulled health information.; The dynamic capture of multi-format, multi organ, multi-time point biodata from the same subject as for within and cross comparators, plus the most robust 360-degree profiling tool to phenotypically characterize the essence of every organ system.

### Chemical Imaging of Molecules in Biosamples

Biomolecular Spectroscopy produces spectral data from the interaction of photons with tissue. Resulting outputs inform the composition and status of the biosample. Spectroscopic chemical imaging covers several scientific fields such as chemometrics; hyperspectral imaging (measuring thousands of contiguous spectral bands); multispectral imaging (measuring spectral bands from different EM regions); and physics-enabled computational data science including machine learning.

‘Spectromics’: this is the non-a self-supervised approach that leverages widely established chemical data without a priori understanding inputs needed to teach or tell the tool based onusing training data defined samples or showcasing conditions. Supervised approaches such as . . . .

The spectral “fingerprint region” (1800-900 cm-1) is known as a source of richly informative and unique collagen-associated molecular spectral signatures of the v(C—O) and v(C—O—C) carbohydrate moieties which demonstrate absorption at 1035 cm-1 and 1079 cm-1 respectively).

. . . diabetic nephropathy (1080 cm-1 and 1030 cm-1). Infrared Molecular Spectroscopy

Kidney Fibrosis lacks a reliable, rapid, reproducible, automated, and precise diagnostic technique. The difficulty in diagnosing renal fibrosis is fibrosis is due in part to the shortcomings of standard pathology methods which are plagued by the unfixable lack nature of standard pathology methods that lack reliability and reproducibility. More specifically, fundamentally discriminating pathologic collagen and constitutive (naturally occurring) collagen is challenging with standard histopathology methods Fibrosis in the Renal Cortex (kidney's outer region) is particularly significant because scarring destroys the glomerular capillaries vital for blood filtration, compromising a key kidney function. When presenting in the cortex, fibrosis not only diminishes kidney function but also causes ischemic damage to other kidney areas, leading to further injury.

Additionally, since the kidney's capillary beds are uniquely connected in series, the loss of these capillaries hinders oxygen and nutrient delivery to downstream peritubular capillaries, which nourish the tubular epithelium in both the cortex and medulla (the kidney's inner part).

For the purpose of our initial use case, organ donation and transplantation, the core needle biopsy attains a sample (1 cm) in length of the cortex. If the medulla is captured, the sample is too deep and therefore inadequate.

Examples of regions where fibrosis is relevant to successful graft function include: interstitium, glomerulus, and arteriole.

Additionally, since the kidney's capillary beds are uniquely connected in series, the loss of these capillaries hinders oxygen and nutrient delivery to peritubular capillaries, which nourish the tubular epithelium in both the cortex and medulla (the kidney's inner part).

When presenting in the cortex, fibrosis not only diminishes kidney function but also causes ischemic damage to other kidney areas, leading to further injury.

Therefore, fibrosis is a strong predictor of long-term kidney outcomes in transplantation and various kidney diseases.

IR spectroscopy is the measurement of varied absorption across the IR spectrum as a function of the vibrational modes of the biomolecules present within a sample.

This allows for study of the molecular composition of a specimen, which will vary with tissue identity and location; modality of spectroscopy set-up; and disease state. In various implementation the software such as ENVI-ILD and OPUS specification inputs of the componentry setup, and conversion calculators for transposing scales depending on modality employed (e.g. FT-IR, QCL-IR, DF-IR) to accommodate variation, standardize and report results per the nature of investigation.

In contrast to existing systems, the biopsy interrogation module employs Discrete Frequency QCL IR laser excitation to enable machine learning model trained using these modalities which honed to more precisely distinguish the viability factors of interest (e.g., fibrosis, sclerosis, and so forth).

IR signals characteristic of:
carbohydrates: (1000-1150 cm-1)
lipids (1150-1200 & 1330-1340 cm-1)
proteins (1240-1300 cm-1)
free amino acids (1396 cm-1)

Spectral profiles reveal dominant analytes of Blood are free amino acids & lipids; Internal vessel walls are lipids & proteins; External walls are proteins; and Glomeruli & Tubulesare glycogen;

Important to note: constitutive collagen is exclusively located in the capsula, walls of vessels, Bowman capsule, and vessel support-blades. So the “geography” of collagen (both natural constitutive & fibrotic) is as important as identifying and quantifying the spectra. The production of ‘biological metadata’ from spectral data extracted tissue substructures. For example, such data are required to both identify the chemical profile to highlight the parts of tissue (e.g blood vessels) compared with all other analytes and of tissue and to reverse engineer using this chemical profile to reconstruct the solid object formed beginning with the understanding of blood vessels and then implementing an analytical strategy

FIG. 5B is a schematic diagram of the optical paths and structures of the biosample analysis station 5000.

FIG. 5C is a perspective drawing of a cartridge 5200 for organ samples. FIG. 5E is a cross sectional drawing of the cartridge 5200;

The cartridge contains a sample (5202), which the cartridge sandwiches between two coated windows such as Barium fluoride “Low-E slides” IR reflectance comes from two layers of silver; QCL-IR imaging (5204) when closed. The spacing of the windows 5204 when closed is calibrated such that for a particular gage of biopsy needle the sample is firmly squeezed on both sides with no air gap between it and the sapphire windows 5204 The sapphire windows (5204) attach to the base (5206) and cover (5208) of the cartridge.

Sapphire has a nearly constant refraction index for light wavelengths from visual to infrared, thus minimizes optical distortion of the combined wavelength beam and allows visual spectrum cameras to also work. The base also features an O-ring gland which fits an O-ring to achieve a hermetic seal of the biopsy sample when the cartridge is closed-preventing cross contamination. The base and cover (connect via a hinge. They also feature a locking device or devices that keep the cartridge closed and sealed during scanning. The cover features visual identification markings such as the serial number that are machine and human readable. There is also an indication of which biopsy needle gauge the cover is for.

FIG. 5F is a schematic flow chart diagram of a method of imaging an ex vivo organ to create a detailed volumetric map, according to one or more examples of the disclosure. The method involves a series of steps to ensure precise imaging and data collection for the ex vivo organ.

Starting with the initiation of the method, in some implementations, the method 5300 includes accepting the organ into the imaging bag. This ensures the organ is securely positioned for imaging. In various implementations, the method 5300 includes waiting for user input to start imaging, ensuring that the process begins only when the operator is ready.

In certain implementations, the method 5300 includes checking if the user input to start has been received. If no input is received, the process waits. If input is received, in one or more implementations, the method 5300 includes closing (5310) the laser protection lid to ensure safety during imaging.

In some implementations, the method 5300 includes measuring and recording the weight of the organ. This data is crucial for subsequent analysis. In various implementations, the method 5300 includes taking (5314) a digital photograph of the organ to document its initial condition.

In certain implementations, the method 5300 includes executing (5316) a surface scan of the organ and solution. This scan provides detailed surface profile data. In one or more implementations, the method 5300 includes computing (5318) the volume of the organ from the surface profile and solution level.

In some implementations, the method 5300 includes uploading (5320) the data obtained so far. This ensures that all collected data is stored securely. In various implementations, the method 5300 includes performing (5322) an initial organ assessment on the organ viability analysis engine 6000 to analyze the preliminary data.

In certain implementations, the method 5300 includes checking (5324) if the organ passed the initial assessment. If the organ does not pass, the process ends. If the organ passes, in one or more implementations, the method 5300 includes generating (5324) an imaging plan with the organ viability analysis engine 6000.

In some implementations, the method 5300 includes downloading (5326) the imaging plan from the organ viability analysis engine 6000. This plan guides the detailed imaging process. In various implementations, the method 5300 includes using (5328) laser head positioning and beam steering to aim the beam at the organ with the proper incident angle and location.

In certain implementations, the method 5300 includes firing (5330) the laser pulse and sending the synchronization signal to the DAQ. This step initiates the detailed imaging. In one or more implementations, the method 5300 includes recording (5332) timed transducer array signals with the DAQ.

In some implementations, the method 5300 includes checking (5334) if other wavelengths need to be studied at the current point. If yes, in various implementations, the method 5300 includes switching (5336) the optical multiplexer to the next laser.

In certain implementations, the method 5300 includes checking (5338) if there are other positions to scan in the program. If yes, the process loops back to re-position the laser head. If no, in one or more implementations, the method 5300 includes uploading (5340) the data to the organ viability analysis engine 6000 for comprehensive analysis.

In some implementations, the method 5300 includes opening (5342) the lid and indicating the status, concluding the imaging process. This method ensures that the ex vivo organ is thoroughly and accurately imaged, maintaining its integrity throughout the process, and providing comprehensive data for informed medical decisions.

FIG. 5G describes the automated programmatic process by which a sample is determined adequate. As an example, this process shows the steps that happen internally following a transfer of the core kidney tissue sample from a core needle (such as a Bard Max-Core Disposable Core Biopsy Instrument) to the proprietary device cartridge. This process starts 5400 when lasers, sensor optics are deemed correctly calibrated 53404 and the machine is ready to perform biopsy 5404 following the cartridge being inserted and accepted into the station 5406 and a start command is issued 5408 If yes, then receiver moves to visual imaging position 5410 then the receiver is moved to the visual imaging position 5410, then sample is assessed for adequacy 5412 In the example provided, a kidney core needle sample adequacy assessment is demonstrated.

The automated adequacy determination process uses a sensor camera in one step to measure the length of the sample by asking if the sample the right length (˜1 cm); if yes then, the process asks the sensor if the sensor detects if the sample is the right color (correct color=coral/transparency variation of the color red); if yes, then the process asks if the sensor camera detects the correct geometric pattern (correct pattern=circular shapes); if yes, then the process asks the sensor camera to count the number of circular shapes detected in the sample (correct number=minimum of at least 15 circular shapes).

If all five binary outputs are ‘yes’ the screen interface (1026) illuminates and the words ‘adequate sample received’ appears 5414. If less than five data outputs were ‘yes’, on the interface appears a tabular output matrix 6 rows, four columns displaying step number, description of step performed, output ‘yes’ or ‘no’, and the final column reason for ‘no’ decision. The screen interface (1026) has touchscreen options on the tabular output that offer the operator a choice to explore further any of the steps executed and images with text rationale are provided if touchscreen selection is made by the operator.

FIG. 5H is a side view drawing of an organ sample 5200;

FIG. 5I describes the process of classification, segmentation, and visualization of a sample via molecular spectroscopy 5500 using chemical response parameters in the wavenumber range commensurate with the laser tuned frequency range. Different methodologies such as supervised and self-supervised and semi-supervised can be used independently and in combination. In the case of the setup described 5100 which employs quantum cascade laser diodes to emit laser beams in the midinfrared region to leverage the information known from the “fingerprint region” Raw, unprocessed spectral data in the midinfrared region is driven by molecular identifiers detected in biological samples such as tissue.

Frequency components can be identified and pulled out automatically using components to determine which frequencies were absorbed. Various preprocessing techniques to mitigate noise, eliminate outliers, and normalize graphic outputs to simplify the data visualization, provide descriptive statistics, classify according to parameters, to produce maps 5502 and analyze biological information relevant for features tied to disease detection.

Supervised approaches enable preprocessing such as principle component analysis (PCA) to determine parameters that explain the most variation in the outcome of interest in the classification and hierarchical cluster analysis (HCA) 5506 clusters substructures based on their features such as chemical composition using prior knowledge to guide spectral analysis, for example when reconstructing tissue samples on the basis of spectral responses of substructures which requires a supervised technique to co-register between the anatomical visualization of the sample and its chemical characterization by matching voxels to their corresponding to the substructure.

To reiterate, as depicted in FIG. 5I which is a schematic flow chart diagram of a method 5500 of spectral analysis of biologic samples to create detailed diagnostic reports, according to one or more examples of the disclosure. The method 5500 involves a series of steps to segment, classify, and analyze spectral data from biologic samples.

Starting with the initiation of the method, in some implementations, the method 5500 includes segmenting and classifying (5502) spectral data of the biologic samples into a mapped format. This step ensures that the data is organized for subsequent analysis. In various implementations, the method 5500 includes clustering (5504) the segmented and classified spectral data to form spectral clusters. These clusters group similar spectral characteristics for more detailed examination.

In certain implementations, the method 5500 includes analyzing (5506) the spectral clusters to extract analytic data. This analysis identifies key features and patterns within the spectral data. In one or more implementations, the method 5500 includes converting (5508) the analytic data into spectral curves represented as images. These images visually represent the spectral characteristics for easier interpretation.

In some implementations, the method 5500 includes compiling (5510) the spectral curves into diagnostic reports that summarize the spectral analysis results. These reports provide a comprehensive overview of the findings, facilitating informed medical decisions. This method ensures that biologic samples are thoroughly analyzed, and the results are presented in a clear and actionable format.

FIG. 5J is an example of how molecular spectroscopy can take classified components and subcomponents (e.g. constitutive and fibrotic collagen) relevant to tissue type based on their spectral response peaks depicted in FIG. 5K to parse and map to a sample image map such as is portrayed here (sourced from a core needle kidney biopsy sample acquired from the cortex of the kidney).

FIG. 5L depicts a working example images to illustrate integration of hyperspectral biosample analysis station 5000 with machine vision in organ viability analysis engine 6000

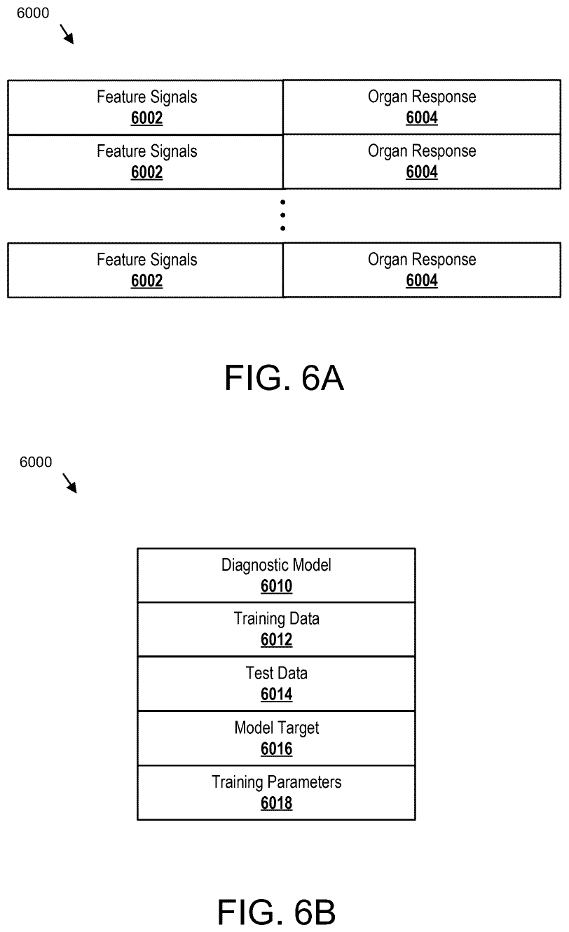

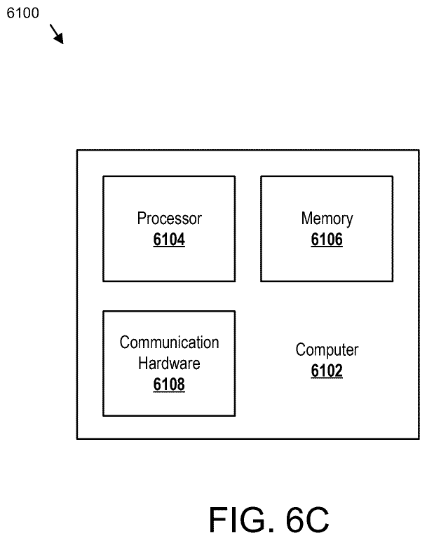

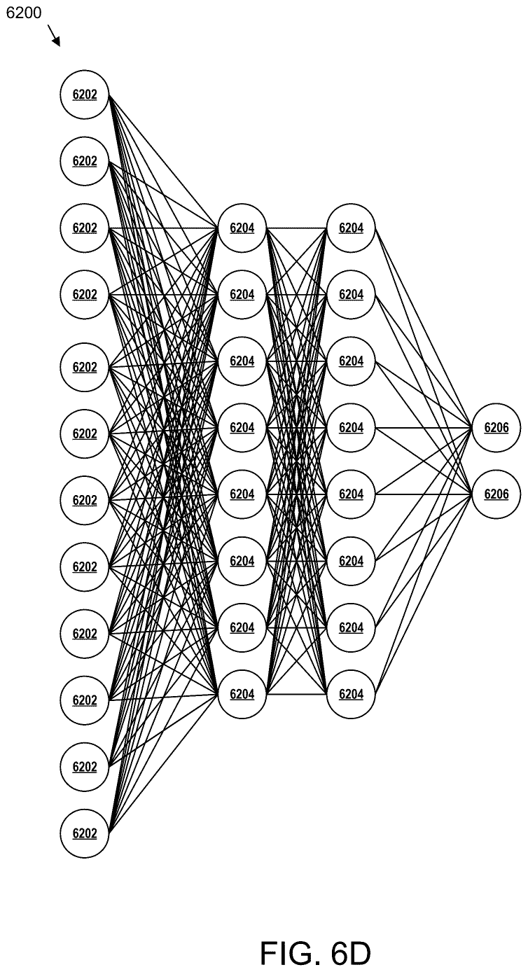

FIG. 5L also illustrates a beneficial capability of the hyperspectral biosample analysis station 5000 depicted in FIG. 5A in communication with the organ viability analysis engine 6000 depicted in FIG. 1 and described with respect to FIGS. 6A-6D.

This capability pertains to the handling and analysis of photomicrograph images, specifically in the context of kidney sample analysis.

Image 5518 on the left side of FIG. 5L represents an initial photomicrograph taken by optical cameras 5002 of a kidney sample. The images displayed are generated via sophisticated imaging techniques that provide high-resolution insights into the microscopic structure of the kidney tissue.

As depicted in Image 5518, the Organ Viability Analysis Engine 6000 utilizes its machine vision capabilities to detect structures within the kidney, specifically the glomeruli, labeled as 5522. These glomeruli are essential microscopic structures in the kidney involved in the filtration process.

Progressing to image 5520 on the right side of FIG. 5L, the capability of the system to overlay boundaries around detected glomeruli is illustrated. The machine vision algorithms process the initial image data to delineate the contours of each glomerulus, enhancing the clarity and focus on these critical structures.

Within this process, the system automatically overlays the boundary as depicted in Image 5520 and counts the detected glomeruli, now identified as 5524. This count is essential for quantitative analysis in medical research and clinical diagnostics, providing a baseline for assessing the health and functionality of the kidney.

Further, the organ viability analysis engine 6000 assesses these glomeruli for pathological conditions based on the size and shape of their perimeters. Pathological glomeruli can be identified and differentiated from healthy ones by analyzing variations in their geometric properties, which are often indicative of diseases such as glomerulonephritis or diabetic nephropathy.

FIG. 5L depicts the capacity to automate a diagnosis such as glomerulosclerosis by measuring of the diameters and shapes of glomeruli to compare and distinguish visible characteristics and features based on size (diameter) 5704 and shape 5706 and classify groups as normal as compared to averages on the basis of regularity in a biosample such as a kidney tissue by activating a computer vision process with the hyperspectral sensor of the hyperspectral hyperspectral biosample analysis station The integration of these imaging and analytical capabilities in the hyperspectral biosample analysis station 5000 and the organ viability analysis engine 6000 showcases the advanced technology available for organ viability assessments. This integration allows for a more thorough and nuanced understanding of organ health, contributing to improved diagnostics and treatment strategies.

FIG. 6A is a schematic block diagram illustrating one embodiment of model data 6000. The model data 6000 may be organized as a data structure in a memory. In the depicted embodiment, the model data 6000 includes a plurality of feature signals 6002 and corresponding organ responses 6004.

A feature signal 6002 may comprise at least one of chemically-derived, molecularly-derived, and/or spectrally-derived signals. Each feature signal 6002 may comprise at least one measurement. Table 1 lists measurements that may be included in the feature signal 6002. Any combination of measurements may be employed in the feature signal 6002.

TABLE 1

acoustic signals
organ weight
organ elasticity
organ volume
three dimensional organ surface
organ image
laser frequency for the laser beam
spatial coordinates of a pathway aperture of the optical pathway
microscopic view
spectral response curves
laser spectroscopy
chemometrics
multi-omics

Volumetric and/or positional information may be defined as voxels, three-dimensional coordinates, and the like.

The acoustic signals may be induced by a laser beam at a specified wavelength. The acoustic signals may be captured by a transducer array in response to the laser beam being applied to via an optical pathway to an organ. The organ may be a kidney or the like.

The organ weight may be a mass of the organ. The three-dimensional organ surface may specify the surface of the organ. The organ volume may be calculated from the three-dimensional organ surface.

The organ image may include one or more images of the organ. The organ image may be captured in infrared light, visible light, ultraviolet light, or combinations thereof.

The laser frequency specifies at least one wavelike frequency of the laser beam that induce the acoustic signals. The spatial coordinates of the pathway aperture of the optical pathway specifies the location and/or orientation of an aperture of the optical pathway of the laser beam.

Each organ response 6004 may comprise at least one result. Table 2 lists results that may be included in the organ response 6004. Any combination of results may be employed in the organ response 6004.

TABLE 2

diagnostic score
hemoglobin map
chromophore distribution
oxygenation map
deoxygenated hemoglobin map
glom proteins
free animo acid proteins
collagen map
fibrosis spectrum
fibrosis centroid
collagen spectrum
collagen centroid

The fibrosis centroid may represent an absorbent frequency for collagen that is fibrotic. The oxygenation map may identify an extinction coefficient. The collagen map may identify molecular vibrations associated with disease. A high ratio of Glom proteins to free animo acid proteins is indicative a low probability of disease.

FIG. 6B is a schematic block diagram of the diagnostic model 6010 and other data. The data may be organized as a data structure in a memory. The diagnostic model 6010 is used to generate an organ response 6004 from feature signals 6002. The diagnostic model 6010 may be trained using the model data 6000 of FIG. 4A. In one embodiment, the model data 6000 is divided into training data 6012 and test data 6014. The diagnostic model 6010 may be trained using the training data 6012. In addition, the diagnostic model 6010 may be trained based on the training parameters 6018.

The test data 6014 may be used to test the diagnostic model 6010. In one embodiment, the feature signals 6002 of the test data 6014 are applied to the diagnostic model 6010. The organ responses 6004 generated by the diagnostic model 6010 are compared to the organ responses 6004 of the test data 6014 and a number of matches determined. The diagnostic model 6010 may be validated if the percentage of matches exceeds the model target 6016.

FIG. 6C is a schematic block diagram illustrating one embodiment of a computer 6102. The computer 6102 includes a processor 6104, a memory 6106, and communication hardware 6108. The memory 6106 may store code and data. The processor 6104 may execute the code and process the data. The communication hardware 6108 may communicate with other devices.

FIG. 6D is a schematic block diagram illustrating one embodiment of a neural network 6200. In the depicted embodiment, the neural network 6200 includes input neurons 6202, hidden neurons 6204, and output neurons 6206. The neural network 6200 may be organized as a convolutional neural network, a recurrent neural network, long short term memory (LSTM) network, U-Net architecture, transformer, and the like.

For example, in some implementations, a U-Net architecture may be advantageously employed by the.

The U-Net architecture is an advanced convolutional neural network designed specifically for the precise segmentation of biomedical images. It features a distinctive U-shaped structure that enables precise localization and a high degree of context retention in image analysis. This architecture is structured in two primary pathways: the contraction (downsampling) path and the expansion (upsampling) path.

The contraction path is composed of a series of convolutional and max pooling layers, which work to capture the context of the image. This path helps the network understand what is present in the input image by reducing its spatial dimensions while increasing the depth, capturing features at various scales.

The expansion path, on the other hand, consists of a series of upconvolution and concatenation steps followed by regular convolutional layers. This path enables precise localization by using transposed convolutions to project feature representations to higher resolution spaces. The critical feature of U-Net is the skip connections that bridge layers of the same size in the contraction and expansion paths. These connections transfer contextual information directly across the network, helping to preserve spatial hierarchies and improve the clarity of the output segmentation.

In certain implementations, the organ viability analysis engine 6000, the U-Net architecture is employed to segment key structural and pathological features within complex organ images. By effectively distinguishing different tissue types and pathological markers in these images, U-Net supports the engine's capability to analyze and assess organ viability with high accuracy and detail.

The neural network 6200 may be trained with the training data 6012. The training data 6012 may include the feature signals 6002. In addition, the training data 6012 may include the organ response 6004. The neural network 6200 may be trained using one or more learning functions while applying the training data 6012 to the input neurons 6202. In one embodiment, a known result organ response 6004 is applied to the output neurons 6206. Subsequently, the neural network 6200 may receive actual feature signals 6002 at the input neurons 6202 and make predictions at the output neurons 6206 based on the actual data.

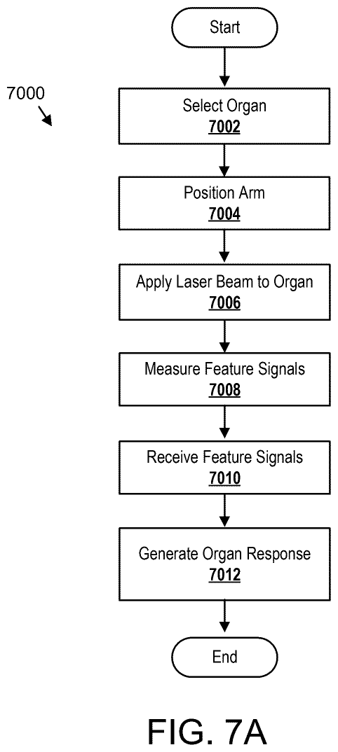

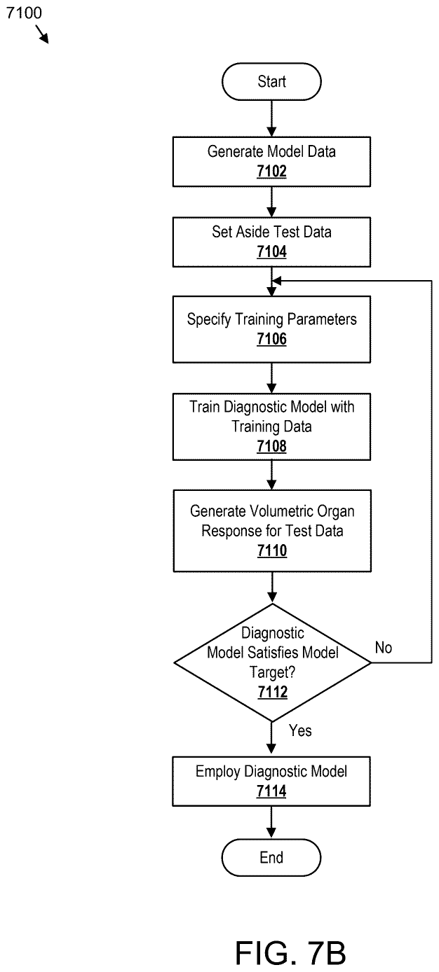

FIG. 7A is a flow chart diagram illustrating one embodiment of the diagnostic method 7000. The diagnostic method 7000 may generate an organ response 6004 for an organ. The method 7000 may be performed by the processor 6104 and/or the neural network 6200.

The method 7000 selects 7002 an organ for analysis. In one embodiment, an interface screen displays a human form with an outline of at least one organ. A user may select an organ outline to select 7002 the organ. In addition, a menu of organs may be displayed and the user selects 7002 the organ from the menu.

The method 7000 positions 7004 the arm (3400). The arm (3400) may be positioned based on programmed geometries using known measurements such as datasets in an x-y-z format of spatial positioning. The arm (3400) may generally localize and indicate the arm (3400) has localized itself above the body with the transducer array aligned in the general area over a region such as the abdominal or cardiothoracic region. Positioning may be semi-automated and/or automated using spatial and/or shape identification of the organ based on photo acoustics such as ultrasound. Once the arm (3400) is positioned 7004 at the correct region/position, the screen may display the arm (3400) and/or organ and the user may confirm the position.

Once the organ of interest is found and indicated on screen, the method 7000 may calculate geometrically the location in 3-dimension proportional location relative to the body as a whole and/or within the cavity. In one embodiment, the position is outside of a body. The position datapoints will be stored along with data from the health record. The health record may include demographic information such as at least one of ethnicity, age, gender, height, weight, body mass index (BMI), race at the like. The health record may be included in subsequent model data 6000 and used to train the diagnostic model 6010 so that over time the diagnostic model 6010 will know to direct the arm (3400) to any selected organ.

The method 7000 applies 7006 a laser beam to the organ. The organ may be an ex vivo organ, a transplant organ, an in vivo organ, organ tissue, or the like. The laser beam may be at a specified wavelength. The laser beam induces acoustic signals. The acoustic signals are captured by the transducer array.

The method 7000 measures 7008 feature signals 6002 for the organ. The feature signals 6002 may include at least one of the measurements of Table 1.

The method 7000 receives 7010 the feature signals 6002. The feature signals 6002 may be communicated to the processor 6104 and/or diagnostic model 6010 executing on the processor 6104 and/or neural network 6200.

The method 7000 generates 7012 the organ response 6004 from the feature signals 6002 using the diagnostic model 6010 and the method 7000 ends. The feature signals 6002 are input as to the diagnostic model 6010 and the organ response 6004 is an output from the diagnostic model 6010. The organ response 6004 comprises at least one measurement from Table 2.

In one embodiment, the feature signals 6002 define one or more voxels for the organ. A co-registration between the voxels and chemical data is established, creating a chemical profile for the organ. In one embodiment, the co-registration is between sub-surface voxels and/or sub-surface structures and the chemical data.

The organ response 6004 may be generated 7012 using supervised analysis for known chemical species, expected chemical species, and or biological metadata of interest.

In addition, the organ response 6004 may be generated 7012 using unsupervised analysis, wherein the feature signals 6002 are analyzed for differences between samples and/or sample compartments. In one embodiment, both supervised and unsupervised analysis are performed and the results are combined in the organ response 6004.

In one embodiment, spectra delineate substructures of voxels into finite families or ranges to form clusters which determine sections defined by their relatively common chemical profiles. The substructures may be members of the same spectral family.

The raw spectra exhibit curve intensities that can be identified and/or extracted in automated fashion. For example, the intensity of absorptions for infrared intensities may be identified and extracted.

In one embodiment, a whole set of spectra is clustered into highly homogeneous spectra families. A mathematical model per cluster may be defined for the cluster.

In one embodiment, a Partial Least Squares (PLS) regression analysis with dimensionality reduction is performed. The PLS regression analysis may be performed according to parameters such as blood, free amino acids, carbohydrates, lipids, proteins.

In addition, the PLS regression analysis may be performed without dimensionality reduction as in random forests (RF) analysis. The RF analysis may classify spectra and works effectively on unprocessed data. RF analysis is driven by randomly sampling spectral features and using these to generate a set of decision trees that vote on the correct classification. One benefit of RF analysis is it can capture a multimodal distribution, which can commonly occur if a single class contains multiple chemical components.

In one embodiment, Monte Carlo analysis is employed to sample the spectrum. These methods can be applied quickly to large data sets without dimension reduction.

FIG. 7B is a flow chart diagram illustrating one embodiment of a diagnostic model training method 7100. The diagnostic model training method 7100 trains the diagnostic model 6010. The method 7100 may be performed by the processor 6104 and/or the neural network 6200.

The method 7100 starts and generates 7102 the model data 6000. The model data 6000 may be generated 7102 from a plurality of measurements of organs. In addition, the model data 6000 may include synthetic data. In one embodiment, the model data 6000 includes a health record.

In one embodiment, the method 7100 sets aside 7104 a portion of the model data 6000 as test data 6014. A specified number of feature signal 6002 and corresponding organ response 6004 pairs may be set aside 553. The model data 6000 that is not set aside 7104 may be the training data 6012.

The method 7100 may specify 7106 the training parameters 6018 for training the diagnostic model 6010. The training parameters 6018 include a model size, a prompt type, a temperature, a maximum token limit, a frequency penalty, a presence penalty, and a top-p. The training parameters 6018 may include a weight for each measurement of a feature signal 6002. In addition, the training parameters 6018 may include a bias for each result of the organ response 6004.

The method 7100 trains 7108 the diagnostic model 6010 with the training data 6012. The feature signals 6002 may be iteratively applied to the neural network 6200 subject to the training parameters 6018 and/or the corresponding organ responses 6004. The neural network 6200 may be iteratively adjusted based on the feature signals 6002 and/or organ responses 6004 to train the diagnostic model 6010.

The method 7100 tests the train diagnostic model 6010 by generating 7110 organ responses 6004 for each feature signal 6002 the test data 6014. The generated organ response 6004 is then compared to the corresponding organ response 6004 of the feature signal 6002 to determine if the generated organ response 6004 and a corresponding organ response 6004 agree and/or match. The agreement may be to within a specified agreement threshold.

The method 7100 determines 7112 whether the trained diagnostic model 6010 satisfies the model target 6016. If the diagnostic model 6010 does not satisfy the model target 6016, the method 7100 moves to specify 7106 new training parameters 6018. For example, the temperature training parameter may be lowered. The method 7100 then re-trains the diagnostic model 6010.

If the method 7100 determines 7112 the diagnostic model 6010 satisfies model target 6016, the diagnostic model 6010 is employed 7114 and the method 7100 ends.

Clauses describing various implementations or embodiments of the present disclosure are provided below.

Clauses describing various implementations or embodiments of the present disclosure are provided below.

1: An The apparatus may include: an organ viability analysis instrument may include: an in situ organ interrogation module may include a positioned photoacoustic array with predetermined spectral and directional laser excitation and acoustic response signal capabilities selected based on an organ type and respective viability factors for an organ in its natural location within a body by performing photoacoustic computational imaging, the photoacoustic array configured to produce one or more first sets of organ response data related to the viability factors for the in situ organ; an ex vivo organ interrogation module may include: a stationary photoacoustic array with predetermined spectral and directional laser excitation and acoustic response signal capabilities selected based on organ type and respective viability factors for an ex vivo organ received in an acoustic coupling nest, the photoacoustic array configured to produce one or more second sets of organ response data related to viability factors by performing photoacoustic, multispectral scanning; and one or more direct measurement sensors configured to perform one or more of gravimetric, dimensional, and environmental measurements related to viability factors for ex vivo organ using respective non-wave-based modalities.

2: The apparatus as paragraph 1 describes, further may include a stabilizer arm configured to couple the photoacoustic array of the in situ organ to a body region selected to facilitate communication of photoacoustic signals between the in situ donor organ and the photoacoustic array, where the stabilizer arm is configured to electromechanically secure the photoacoustic array in a stabilized position in response to determining based on organ response data, that the photoacoustic array is geometrically oriented to enhance organ response to in situ organ photoacoustic interrogation for the selected organ type.

3: The apparatus as either of clauses 1 or 2 describe, further may include: an AI-enabled positioning system integrated with the stabilizer arm, configured to calculate and store the 3-dimensional coordinates of the stabilizer arm's positioning within a body cavity relative to predefined organ locations; a data storage unit configured to store the calculated 3-dimensional coordinates alongside Electronic Health Records (EHR) including one or more patient demographics selected from height, weight, Body Mass Index (BMI), and race; where the AI-enabled positioning system utilizes the stored data to autonomously identify the precise locations of up to eight different organs within the body cavity at the push of a button, based on the collected demographics and previous positioning data, to enhance the apparatus's ability to perform targeted organ analysis with minimal setup time.

4: The apparatus as any of clauses 1-3 describe, further may include: a plurality of wheels; and a handle coupled to the stabilizer arm and may include one or more hand grips configured to enable a console of the instrument to be tilted for transport by rolling.

5: The apparatus as any of clauses 1-4 describe, further may include one or more console support rests that secure stable positioning of the instrument in a horizontal orientation,

6: The apparatus as any of clauses 1-5 describe, further may include an environmental controller that maintains the environment of the ex vivo organ received in the acoustic coupling nest within a predetermined range of environmental parameters that are based on the organ type.

7: The apparatus as any of clauses 1-6 describe, further may include a hyperspectral biosample analysis station configured to spectroscopically characterize one or more spectral parameters related to the organ viability factors for the selected organ type.

8: The apparatus as any of clauses 1-7 describe, where the hyperspectral biosample analysis station may include one or more discrete frequency quantum cascade laser diodes configured to perform spectroscopic scanning of a biopsy sample to spectroscopically scan the biopsy sample using spectral interrogation parameters to distinguish pathological organ components from constitutive organ components based on the selected organ type.

9: The apparatus as any of clauses 1-8 describe, where the organ type is a kidney, the hyperspectral biosample analysis station is configured to spectroscopically determine a fibrotic status, a sclerotic status, or a combination thereof based on respective spectroscopic organ responses that enable a comparison of pathologic collagen to constitutive collagen present in the biopsy sample.

10: The apparatus as any of clauses 1-9 describe, further may include a controller configured to programmatically adjust scanning coordinates and timing of spectroscopic measurements of the biopsy sample.

11: The apparatus as any of clauses 1-10 describe, further may include a cartridge may include selectively spectrally transmissive windows that: permit spectroscopic scanning of the biopsy sample; and reduce risk of exposure to potentially injurious laser emissions.

12: The apparatus as any of clauses 1-11 describe, where the hyperspectral biosample analysis station is configured to be: in data communication with the organ viability instrument; and mechanically separable from the organ viability instrument.

13: The apparatus as any of clauses 1-12 describe, further may include an organ viability analysis engine configured to output organ viability factor values based on input derive from collective organ responses produced by the in situ organ interrogation module, the ex vivo organ interrogation module, and the spectral biopsy analyzer.

14: The apparatus as any of clauses 1-13 describe, where the organ viability analysis engine may include one or more of signal preprocessing functions, feature extraction, and machine learning algorithms; where the signal preprocessing functions are configured to normalize, filter, and denoise data received from the in situ organ interrogation module, the ex vivo organ interrogation module, and the hyperspectral biosample analysis station; the feature extraction is configured to identify and isolate key characteristics from the preprocessed signals that are indicative of organ health and viability; and the machine learning algorithms are configured to analyze these features to generate a predictive model that assesses a predetermined set of organ viability factors for the organ based on historical outcomes and real-time data comparisons.

15: The method as any of clauses 1-14 describe, further may include: identifying, via the machine learning algorithms, parameters and hyperparameters that are most crucial in explaining the outcome of interest, such as a diagnosed disease; where the identification may include analyzing the importance of features derived from the signal preprocessing, feature extraction, and their influence on the predictive model's accuracy and reliability in diagnosing the disease; and where the machine learning algorithms are further configured to adjust the weighting of identified crucial parameters and hyperparameters to optimize the predictive model for enhanced diagnostic performance based on historical data and real-time analysis.

16: The method as any of clauses 1-15 describe, where the feature signals 6002 further may include at least one of chemically-derived, molecularly-derived, and/or spectrally-derived signals for at least one of an organ weight, organ elasticity, organ volume, a three dimensional organ surface profile, an organ image, a laser frequency for the laser beam, the optical pathway for the laser beam, spatial coordinates of a pathway aperture of the optical pathway, microscopic view, and spectral response curves.

17: The method as any of clauses 1-16 describe, where the acoustic signals are induced by the laser beam at a specified wavelength.

18: The method as any of clauses 1-17 describe, where: the acoustic signals are induced by a laser beam at wavelengths specifically selected at 680 nm, 725 nm, and 755 nm within the Q-band and Soret-band spectral regions, identified as optimal for estimating the concentration of collagen in the presence of oxyhemoglobin (HbO) and deoxyhemoglobin (Hb); the method includes quantifying collagen, the primary protein in fibrosis, in conjunction with determining the concentrations of HbO and Hb, employing the extinction coefficients for collagen, deoxyhemoglobin, and oxyhemoglobin within the spectral range of 680-930 nm; where this quantification process is utilized to adjust the diagnostic assessment of fibrotic conditions in biological tissues, enhancing the precision of pathology evaluation by optimizing the spectral interrogation parameters based on the light-absorption characteristics of these biomolecules.

19: The method as any of clauses 1-18 describe, further may include: employing an unsupervised spectral unmixing algorithm configured to detect and differentiate deoxyhemoglobin (Deoxy Hb) and oxyhemoglobin (Oxy Hb) from type I collagen within a spectral range of 680-900 nm, corresponding to the Q band and Soret band regions; where the spectral unmixing algorithm operates without prior knowledge of the specific spectral signatures of the substances, enhancing its capability to parse and quantify the presence of Deoxy Hb, Oxy Hb, and type I collagen in kidneys exhibiting fibrosis; and where the algorithm analyzes the acoustic signals induced by the laser beam, adjusted to the specified wavelength within the 680-900 nm range, to determine the concentration and distribution of these biomolecules as a measure of organ health and pathology.

20: The method as any of clauses 1-19 describe, where each organ response further may include at least one of a chromophore distribution, an oxygenation map, a deoxygenated hemoglobin map, and a collagen map.

21: The method as any of clauses 1-20 describe, where the organ response is combined with a health record.

22: The apparatus as any of clauses 1-21 describe, where the machine learning algorithms for the predictive model are implemented by: training the predictive model on a plurality of feature signals and corresponding organ responses, where at least a portion of the feature signals may include acoustic signals captured by a transducer array in response to a laser beam applied via an optical pathway to a specified organ of a plurality of organs and a specified corresponding organ response for the feature signals may include a diagnostic score; measuring selected feature signals for the selected organ type; and generating an organ response from the selected feature signals using the predictive model.

Examples and implementations may be practiced in other specific forms. The described examples are to be considered in all respects only as illustrative and not restrictive, unless otherwise clear from context. The scope of the invention is, therefore, indicated by the appended claims rather than by the foregoing description. All changes which come within the meaning and range of equivalency of the claims are to be embraced within their scope.

What is claimed is:

1. An apparatus comprising:
an organ viability analysis instrument comprising:
an in situ organ interrogation module comprising a positioned photoacoustic array with predetermined spectral and directional laser excitation and acoustic response signal capabilities selected based on an organ type and respective viability factors for an organ in its natural location within a body by performing photoacoustic computational imaging, the photoacoustic array configured to produce one or more first sets of organ response data related to the viability factors for the in situ organ;
an ex vivo organ interrogation module comprising:
a stationary photoacoustic array with predetermined spectral and directional laser excitation and acoustic response signal capabilities selected based on organ type and respective viability factors for an ex vivo organ received in an acoustic coupling nest, the photoacoustic array configured to produce one or more second sets of organ response data related to viability factors by performing photoacoustic, multispectral scanning; and
one or more direct measurement sensors configured to perform one or more of gravimetric, dimensional, and environmental measurements related to viability factors for ex vivo organ using respective non-wave-based modalities.

2. The apparatus of claim 1, further comprising a stabilizer arm configured to couple the photoacoustic array of the in situ organ to a body region selected to facilitate communication of photoacoustic signals between the in situ donor organ and the photoacoustic array, wherein the stabilizer arm is configured to electromechanically secure the photoacoustic array in a stabilized position in response to determining based on organ response data, that the photoacoustic array is geometrically oriented to enhance organ response to in situ organ photoacoustic interrogation for the selected organ type.

3. The apparatus of claim 1, further comprising:
an AI-enabled positioning system integrated with the stabilizer arm, configured to calculate and store the 3-dimensional coordinates of the stabilizer arm's positioning within a body cavity relative to predefined organ locations;
a data storage unit configured to store the calculated 3-dimensional coordinates alongside Electronic Health Records (EHR) including one or more patient demographics selected from height, weight, Body Mass Index (BMI), and race;
wherein the AI-enabled positioning system utilizes the stored data to autonomously identify the precise locations of up to eight different organs within the body cavity at the push of a button, based on the collected demographics and previous positioning data, to enhance the apparatus's ability to perform targeted organ analysis with minimal setup time.

4. The apparatus of claim 1, further comprising:
a plurality of wheels; and
a handle coupled to the stabilizer arm and comprising one or more hand grips configured to enable a console of the instrument to be tilted for transport by rolling.

5. The apparatus of claim 1, further comprising one or more console support rests that secure stable positioning of the instrument in a horizontal orientation.

6. The apparatus of claim 1, further comprising an environmental controller that maintains the environment of the ex vivo organ received in the acoustic coupling nest within a predetermined range of environmental parameters that are based on the organ type.

7. The apparatus of claim 1, further comprising a hyperspectral biosample analysis station configured to spectroscopically characterize one or more spectral parameters related to the organ viability factors for the selected organ type.

8. The apparatus of claim 7, wherein the hyperspectral biosample analysis station comprises one or more discrete frequency quantum cascade laser diodes configured to perform spectroscopic scanning of a biopsy sample to spectroscopically scan the biopsy sample using spectral interrogation parameters to distinguish pathological organ components from constitutive organ components based on the selected organ type.

9. The apparatus of claim 8, wherein the organ type is a kidney, the hyperspectral biosample analysis station is configured to spectroscopically determine a fibrotic status, a sclerotic status, or a combination thereof based on respective spectroscopic organ responses that enable a comparison of pathologic collagen to constitutive collagen present in the biopsy sample.

10. The apparatus of claim 1, further comprising a controller configured to programmatically adjust scanning coordinates and timing of spectroscopic measurements of the biopsy sample.

11. The apparatus of claim 1, further comprising a cartridge comprising selectively spectrally transmissive windows that:
permit spectroscopic scanning of the biopsy sample; and
reduce risk of exposure to potentially injurious laser emissions.

12. The apparatus of claim 1, wherein the hyperspectral biosample analysis station is configured to be:
in data communication with the organ viability instrument; and
mechanically separable from the organ viability instrument.

13. The apparatus of claim 1, further comprising an organ viability analysis engine configured to output organ viability factor values based on input derive from collective organ responses produced by the in situ organ interrogation module, the ex vivo organ interrogation module, and the spectral biopsy analyzer.

14. The apparatus of claim 1, wherein the organ viability analysis engine comprises one or more of signal preprocessing functions, feature extraction, and machine learning algorithms; wherein the signal preprocessing functions are configured to normalize, filter, and denoise data received from the in situ organ interrogation module, the ex vivo organ interrogation module, and the hyperspectral biosample analysis station; the feature extraction is configured to identify and isolate key characteristics from the preprocessed signals that are indicative of organ health and viability; and the machine learning algorithms are configured to analyze these features to generate a predictive model that assesses a predetermined set of organ viability factors for the organ based on historical outcomes and real-time data comparisons.

15. The apparatus of claim 14, wherein the organ viability analysis engine performs:
identifying, via the machine learning algorithms, parameters and hyperparameters that are most crucial in explaining the outcome of interest, such as a diagnosed disease;
wherein the identification comprises analyzing the importance of features derived from the signal preprocessing, feature extraction, and their influence on the predictive model's accuracy and reliability in diagnosing the disease;
and wherein the machine learning algorithms are further configured to adjust the weighting of identified crucial parameters and hyperparameters to optimize the predictive model for enhanced diagnostic performance based on historical data and real-time analysis.

16. The apparatus of claim 15, wherein the machine learning algorithms for the predictive model are implemented by:
training the predictive model on a plurality of feature signals and corresponding organ responses, wherein at least a portion of the feature signals comprise acoustic signals captured by a transducer array in response to a laser beam applied via an optical pathway to a specified organ of a plurality of organs and a specified corresponding organ response for the feature signals comprise a diagnostic score;
measuring selected feature signals for the selected organ type; and
generating an organ response from the selected feature signals using the predictive model.

17. The apparatus of claim 15, wherein the feature signals further comprise at least one of chemically-derived, molecularly-derived, and/or spectrally-derived signals for at least one of an organ weight, organ elasticity, organ volume, a three dimensional organ surface profile, an organ image, a laser frequency for the laser beam, the optical pathway for the laser beam, spatial coordinates of a pathway aperture of the optical pathway, microscopic view, and spectral response curves.

18. The apparatus of claim 15, wherein the acoustic signals are induced by the laser beam at a specified wavelength.

19. The apparatus of claim 15 wherein each organ response further comprises at least one of a chromophore distribution, an oxygenation map, a deoxygenated hemoglobin map, and a collagen map.
The apparatus of claim 15, wherein the organ response is combined with a health record.
